(19) United States
(12) Patent Application Publication
(10) Pub. No.: US 2021/0338896 A1
(43) Pub. Date: Nov. 4, 2021

(54) METHOD FOR PRODUCING A MATERIAL FOR A BONE IMPLANT

(71) Applicant: STIMOS GMBH, KONSTANZ (DE)

(72) Inventors: MICHAEL GIESSL, KONSTANZ (DE); HELMUT COELFEN, KONSTANZ (DE); DIETMAR SCHAFFARCZYK, KREUZLINGEN (CH)

(21) Appl. No.: 17/366,111

(22) Filed: Jul. 2, 2021

Related U.S. Application Data
(62) Division of application No. 16/463,593, filed on May 23, 2019.

(30) Foreign Application Priority Data
Nov. 25, 2016 (DE) 102016014052.6
Nov. 26, 2016 (DE) 102016122837.0

(51) Int. Cl. A61L 27/46; C08B 37/08

(52) U.S. Cl. CPC A61L 27/46; C08B 37/0072; A61L 2430/02; A61L 2420/04; A61L 2420/02

(57) ABSTRACT

A material for a bone implant contains: (a) a carrier structure having a surface that has at least one biocompatible material; (b) a matrix covalently bound to the surface; and (c) calcium phosphate embedded in the matrix. A medically acceptable, highly compatible and versatile material can be provided, if the matrix has at least one polysaccharide.

### CROSS-REFERENCE TO RELATED APPLICATION

This application is a divisional of patent application Ser. No. 16/463,593, filed May 23, 2019; which was a § 371 national stage filing of international application No. PCT/EP2017/025344, filed Nov. 24, 2017, which designated the United States; this application also claims the priority, under 35 U.S.C. § 119, of German patent applications No. DE 10 2016 014 052.6, filed Nov. 25, 2016 and DE 10 2016 122 837.0, filed Nov. 26, 2016; the prior applications are herewith incorporated by reference in their entirety.

### BACKGROUND OF THE INVENTION

### Field of the Invention

The present invention relates to a material for a bone implant comprising a carrier structure having a surface that comprises at least one biocompatible material, a matrix covalently bound to said surface, and calcium phosphate embedded in said matrix. The present invention further relates to a method for producing the material according to the invention, a bone implant to which the material according to the invention is applied, and use thereof as a bone implant material.

In recent years, the number of patients suffering from bone damage and/or requiring an implant has tended to increase. This trend highlights the need for research on high-quality, stable and functional bone replacement materials.

The organic components in the bone are composed to 95% of collagen and to 5% of proteoglycans and other adhesion-mediating glycoproteins. The mineral portion of the bone is composed almost exclusively of calcium phosphate in the modified form of hydroxyapatite. The hard material properties of hydroxyapatite, in combination with the elastic properties of the organic components, make bone a highly versatile composite material.

The requirements placed on high-quality and functionally appropriate bone implants are diverse, and it is difficult to meet all of these requirements with one material. The functionality of an implant material is very difficult to predict, as the natural process of bone wound healing and implant healing is highly complex and in some respects not yet fully understood.

The wound healing of hard or soft tissues following surgery such as e.g. bone implantation includes many cellular and extracellular events. The healing process and the contact surface between the bone implant and the bone comprises partially overlapping stages. These comprise inflammation reactions, the formation of a soft callus, followed by formation of a hard callus, and finally the remodeling phase.

Following surgery, proteins and other molecules of the blood and tissue fluids are first adsorbed on the surface of the implant. When vascularized tissue suffers a wound, this causes not only inflammatory reactions, but also activation of numerous further endogenous protective systems such as e.g. the extrinsic and intrinsic coagulation system, the complement systems, the fibrinolytic system and the kinin system. This is followed by two sequential phases that can also temporarily overlap, namely the acute and chronic inflammation reactions. The blood coagulates to form a clot, which is composed of fibrin as its main component. In parallel to this, cytokines and further growth factors are released in order to recruit white blood cells to the wound. In the acute inflammation response, neutrophils and mononuclear cells such as e.g. monocytes are recruited. Mononuclear cell differentiate into macrophages and accumulate on the surface of the implant. In the normal course of wound healing, macrophages are responsible for cleaning the wound by eliminating bacteria, cell debris and other impurities via phagocytosis. During this process, the implant material is also perceived by the body as foreign matter. However, as the implant is much larger than the macrophages, they are unable to phagocytose the material. These events finally lead to the phase of chronic inflammation at the material/tissue interface. Here, the macrophages fuse to form polynuclear giant cells in order to surround the foreign body. The macrophages also recruit further cells such as e.g. fibroblasts, which form fibrous tissue on the surface of the implant.

After the inflammation phase, a soft callus forms. This is composed of bone precursor cells and fibroblasts, which are located in a disordered matrix of non-collagenous proteins and collagen. This matrix is gradually formed by said cells as a first reaction, and is structurally similar to woven bone. The soft callus is finally converted by osteoblasts into an ordered lamellar Bone structure. In this process, osteoblasts secrete type I collagen, calcium phosphate and calcium carbonate having a random, arbitrary orientation. The remodeling phase overlaps with the formation of the hard callus. This occurs by resorption of the disordered bone structures by osteoclasts and subsequent formation of ordered bone structures by osteoblasts.

In order for substances to be suitable for use as bone implant materials and in order to allow optimum healing of the defect site, the substances must show several special properties. Examples of these include biocompatibility, immunogenicity, osseointegration and iatrogenicity. In order to achieve favorable biocompatibility, the material or its degradation products must not be toxic, carcinogenic or teratogenic. No inflammatory, immune, or other negative or unfavorable reactions may be triggered, neither in the environment of the implant nor in the rest of the body. In the event of an immune response, the implant is separated by encapsulation from the rest of the body. The isolation of the connective tissue capsule prevents osseointegration of the implant into the neighboring bone tissue, as the connective tissue constitutes a barrier to the formation of blood vessels and thus also to the necessary transport of oxygen and nutrients. An implant must not trigger an immune response of the body in any case, i.e. it must show no immunogenicity. Particularly important for suitability as an implant material is also favorable osseointegration, by means of which one achieves stable fastening of the foreign material to the bone, which must later also be sufficiently strong for the everyday weight-bearing activities of the patient.

When individual particles come loose from the implant, these may also not cause any of the above-mentioned reactions, and should also either be biodegradable or secretable in order to prevent permanent accumulation and deposition in the body or aseptic loosening of the endoprosthesis. Potential bone materials should therefore show reliable strength, high resilience, high friction resistance, corrosion resistance, and a stiffness similar to that of bone. The latter property plays a particularly important role in the context of so-called “stress shielding.” Bone is a dynamic system that is built up or resorbed. If a bone implant material is used that shows higher stiffness than the bone substance, it takes over the majority of the mechanical load, causing the surrounding bone to be gradually resorbed.

A further criterion is so-called biocompatibility. A potential implant material should be either as bioinert or bioactive as possible. A bioinert material causes no chemical or biological reactions in the body. The implant is therefore biocompatible, and ideally forms a positively locking bond to the bone substance (contact osteogenesis). As a result, only the transfer of a pressure load is possible between the implant and the bone, but this is quite sufficient for many applications, such as the replacement of skull bones, in dental implants, and in the case of fixing pins for bone fractures. This material type includes implants produced from titanium, aluminum, cobalt, chromium and polyether ether ketone (PEEK).

On the other hand, a bioactive material requires rapid growth of the implant into the surrounding tissue and thus provides rapid and long-lasting fixation of the implant in the body. This effect is referred to as so-called “osseointegration.” Bioactive materials therefore often have osteoconductive and osteoinductive properties and usually show high hydrophilicity. Such materials are often resorbed by the body. Hydroxyapatite, tricalcium phosphate and certain bioglasses are included in the bioactive materials. In the worst case scenario, bioactive materials can also trigger an immune response in the body. This capacity of biomaterial to promote cellular adhesion and migration is decisive for the early phases of wound healing and the late phases of bone neoformation and depends to a great extent on the initial contact between the cells and the implant material.

In the prior art, therefore, bone implant materials were coated with hydroxyapatite or tricalcium phosphate by various chemical or physical methods. These calcium phosphates are usually produced using simple chemical methods by means of precipitation from aqueous solutions. Application to the surface of the implant material is carried out either directly from solution onto the surface or by means of physical methods such as “electrospray deposition.” In coating of materials with apatite, however, both the poor adhesion of the calcium phosphates to the implant and their limited cohesion within the individual calcium phosphate layers are disadvantageous. By means of these methods, a structure should be generated on the surface that is as similar to bone as possible in order to promote healing of the material into the bones. Here, however, we are disregarding the fact that the bone itself is a composite material having a highly hierarchical structure that is composed of a matrix and a mineral phase.

Certain methods for coating implant materials with collagen have been investigated for their in vivo functionality. In frequent cases, collagen-coated titanium implants such as screws or nails were analyzed in different in vivo systems. For example, there were reports of positive effects with respect to growth of the material into the surrounding bone and bone neoformation. However, contradictory results were obtained in the prior art with respect to collagen-coated titanium implants. For example, collagen-coated porous titanium cylinders implanted in the diaphysis of the tibia showed no improved osseointegration.

There were also reports in the prior art on methods for the covalent or non-covalent immobilization on surfaces of various proteins of the extracellular matrix such as e.g. fibronectin or short peptides. In some cases, there were positive effects in in vitro test systems such as cellular adhesion and proliferation.

For reasons connected with cost and handling, collagen is often replaced by its denatured form, gelatin. Gelatin is ordinarily produced by physical and chemical degradation or thermal denaturing of native collagen. In contrast to native collagen, gelatin is water-soluble at physiological pH and melts at a sol-gel transition temperature of 25 to 30° C. After cooling, transparent gels are obtained. The non-covalent application of gelatin to the surfaces of arterial implant materials is also reported in the prior art. Gelatin was also covalently coupled to PEEK and mineralized with calcium phosphate in order to form a bone-like layer that led to highly favorable proliferation of osteoblasts. The problem with gel-based coatings is that gelatin is an animal product, which requires class III certification. As gelatin is industrially produced from bovine bone or pigskin, however, there can be religious (pig) or medical objections (cattle, BSE) that can limit the use of a gelatin-based coating.

### BRIEF SUMMARY OF THE INVENTION

On this basis, the object of the present invention is to provide a material for bone implants that is medically safe, highly compatible, and versatile and also has bone-like structures. A corresponding production method is also to be provided.

The object is achieved according to the invention by the features of the independent claims. Favorable embodiments and advantages of the invention are given in the further claims and the description.

In particular, a subject matter of the present invention relates to a material for a bone implant, comprising:

a) a carrier structure having a surface that comprises at least one biocompatible material,
b) a matrix covalently bound to said surface, and
(c) calcium phosphate embedded in said matrix.

It is proposed that the matrix should comprise at least one polysaccharide.

The terms “material for bone implants” and “bone implant material” are used as synonyms herein. The material for bone implants according to the invention has bioactive properties. The term “bioactive” as used herein refers to the property of the material for bone implants according to the invention of allowing rapid growth into the surrounding tissue and thus providing rapid and long-lasting fixation of the implant in the body. This property is derived from the technical features defined in the above subitems (a) to (c) in combination with the characterizing portion.

When bone implants are discussed in the literature, an attempt is always made to make the surface of the implant as similar to bone as possible. In the simplest case, this can be carried out by plasma coating with hydroxyapatite. For example, the method is known of using a gelatin/collagen-based covalently bonded coating that is mineralized with calcium phosphate. This is the most bone-like coating conceivable for implants. In this manner, one can produce a bond for mineralization like that produced in biomineralization (bone and dentin in teeth) by collagen or gelatin as degraded gelatin.

Polysaccharides, in contrast, are not discussed in connection with the biomineralization of calcium phosphate, or if at all, only as glycoproteins. However, polysaccharides play a role in the biomineralization of calcium carbonate (eggshell keratan sulfate, coccolith exoskeletons, etc.), but even in this case, very little research is conducted on them compared to proteins, as polysaccharides are notoriously difficult to characterize.

The approach according to the invention can therefore be seen as a move away from conventional approaches. The idea of using polysaccharides in constructing a bonelike bioinspired coating for an implant is therefore by no means obvious. What would be obvious, as mentioned above, would be to use collagen/gelatin or at least other proteins (here in particular acidic proteins). Surprisingly, however, it has been found that in this application, polysaccharides constitute a well-targeted and advantageous alternative to known substances such as e.g. collagens.

In this context, a carrier structure should be understood to refer to at least one material layer that is composed of the biocompatible material and is bondable or can be covalently bonded to the matrix. The carrier structure can for example be an outermost layer of a basic structure of the implant that is completely produced from the biocompatible material and is for example shapeable. Or it can be applied to a basic structure produced from another material, such as the biocompatible material. Moreover, the term biocompatible is to be understood in this context to refer to the property of a material or substance of being usable in vivo, and also of having few to no adverse effects on the patient or the healing process and being usable without causing any secondary damage.

Here, a matrix is to be understood to refer to a structure that comprises as its main component a polysaccharide in which calcium phosphate is embedded. The polysaccharide can be any polysaccharide considered by the person having ordinary skill in the art to be usable. If the polysaccharide is an animal polysaccharide, a substance can be used whose properties are known and have been tested to a sufficient degree. Advantageously, the polysaccharide is a plant polysaccharide, whereby a vegan substance can be used that is medically safe. In general, it is also possible to use “artificial” polysaccharides or those not occurring in nature. Or it is possible to use mixtures of polysaccharides. These can be selectively produced in the laboratory. Because of their freely designed coating chemistry, such polysaccharides can be selected and used in a highly flexible manner.

Here, the term polysaccharides is also to be understood as referring to—synthetic—substitute materials for polysaccharides, such as e.g. polymers of polyacrylic acid or vinyl and acrylic monomers (see below for possible species). In this context, all features mentioned with respect to the polysaccharides—chemical, material, or relating to a method, a use, or a bone implant—are also to apply in any combination to thesynthetic—substitute material(s).

According to a further embodiment of the invention, it is thus provided that the matrix, instead of the polysaccharide, comprises at least one polymer of polyacrylic acid and/or one polymer of vinyl and acrylic monomers.

For example, the polysaccharide could be alginic acid, alginate, hyaluronic acid, hyaluronate, pectin, carrageenan, agarose, amylose and chitosan. Also possible would be any other glycosaminoglycan, such as heparin/heparan sulfate, chondroitin sulfate/dermatan sulfate or keratan sulfate. Also conceivable are hemicelluloses such as xylans or mannans after carboxyl functionalization, or also xanthan, gellan, fucogalactan or welan gum.

In a preferred embodiment, the polysaccharide is selected from the group consisting of alginic acid, alginate, hyaluronic acid, hyaluronate, pectin, carrageenan, agarose, amylose and chitosan. This allows many different substances to be used, which because of their special properties can be individually selected.

Hyaluronic acid (hya) is a linear polysaccharide that is composed of disaccharide repeating units. These are composed of D-glucuronic acid and N-acetylglucosamine, which are linked via β-1,4 and β-1,3 glycosidic bonds (see below).

In a physiological environment, the carboxyl groups of hyaluronic acid are predominantly in the form of sodium salt, and it is therefore negatively charged and immobilizes a large number of water molecules, with the result that an aqueous solution thereof is highly viscous even at very low concentrations. Hya is used in a wide variety of applications in the medicine and health care industry. As hyaluronic acid is considered to be largely biocompatible and safe, there are numerous areas for its use. This also makes it highly attractive as a starting material for the surface coating of bone implants.

In the past, hyaluronic acid was chiefly extracted from rooster combs as waste from food production. However, production is now carried out biotechnologically by cultivation of genetically modified Streptococcus bacteria, thus eliminating the risk of contamination by animal pathogens.

Alginic acid is a copolymer of the two branched uronic acids D-mannuronic acid (M) and L-glucuronic acid (G). Alginic acid can be obtained from plants or algae, with the result that its composition and the sequence distribution of its sugar units depend strongly on the place of origin and species of the plants/algae used. The G and M respectively are linked via β-1,4-glycosidic bonds. The favorable gelling properties of alginic acid are due to the G-blocks, which complex the calcium ions. As alginic acid is derived from natural sources, one must ensure that potential contaminants such as heavy metals, endotoxins, etc. are removed in order to avoid the quite real possibility of immune reactions. Provided that it has been sufficiently purified, alginic acid, like hyaluronic acid, does not cause any immune or inflammatory reactions in the body, i.e. shows favorable biocompatibility.

In contrast to hyaluronic acid, alginic acid is not degradable in the human body, because no alginase is present. Due to its favorable biocompatibility, alginic acid is used in numerous biomedical applications, for example in wound dressings, and is also suitable, when it is RGD-peptide modified, for use as a bone implant material. When alginic acid is used in combination with hydroxyapatite, the formation of bone tissue can be additionally stimulated.

Moreover, long-chain polysaccharides, but also branched polysaccharides such as e.g. starch, can be used. This gives the matrix a more three-dimensional structure that is similar to the structure of the target bone, thus allowing mineralization to be facilitated.

One possibility of additionally imparting antibacterial properties to the coating/surface processing in addition to its property of bone-identical mineralization (capacity) is the use of chitosan, a polysaccharide-based linear biopolymer: with chitosan, the hemostatic efficacy and antimicrobial properties of the material can also be utilized. Chitosan is also reported to have the following material properties: it is non-allergenic, non-toxic, wound-healing, antibacterial, hemostatic, bacteriostatic, has a fungicidal action (is biodegradable) and is anti-microbial.

The invention thus also relates to chitosan as an individual substance without combination with the above-mentioned compounds or substances. In particular, the invention also relates to the use of chitosan according to the invention as a materialin a medical and/or non-implantable manner and/or with and without contact with the body. In particular, the invention further relates to the use of chitosan according to the invention as a component of a body care product, in particular a toothbrush (toothbrush head), comb, hairbrush, nail brush, nail file, etc. In particular, the invention further relates to the use of chitosan according to the invention as a component of a wellness product, in particular a sleeping mask, a beauty patch, etc. In particular, the invention further relates to the use of chitosan according to the invention as a component of a medicinal product, in particular for wound care (a bandage, plaster, swab, cooling compress, etc.).

If one bonds the polysaccharide using the hydroxyl group present at the 6 position in most polysaccharides via an ester bond to the implant by means of linkers such as succinic anhydride, or bonds it directly to polyacrylic-acid-coated PEEK via an ester bond (optionally by activation chemistry via 1-ethyl-3-(3-dimethylaminopropyl)carbodiimide (EDC) or the like), this makes the entire repertoire of polysaccharides available for covalent bonding to the implant. This also makes it possible to use mixtures of polysaccharides or polysaccharides and synthetic polymers. This in turn makes a completely free composition of the coating available. This can range from negatively charged polysaccharides such as hyaluronic acid to neutral polysaccharides such as amylose to positively charged polysaccharides such as chitosan. This allows the complete spectrum of charge densities, from neutral to strongly negative or positive, to be covered. Of course, this also influences the structure of the coating covalently bonded to the implant and the mineralization of the calcium phosphate. In this process, the mixture of the bonded polymers, and thus their characteristic profiles, can be varied in a stepless manner.

Furthermore, in such mixtures, the structure of the coating can also be adjusted in a selective manner, as for example linear and branched polysaccharides can be combined. For example, branched amylopectin from plant starch can be combined with linear amylose. The two molecules are chemically, but not structurally, identical.

For example, a promising pair of chemically different polysaccharides would be that of neutral amylose with negatively charged alginate, which can additionally be crosslinked by adding Ca²⁺ ions. By means of this crosslinking, further layers of alginates can subsequently be bonded by simple impregnation of the layer with Ca²⁺, washing, and application of the next alginate layer (layer by layer assembly, LBL). An extreme combination would be that of negative alginic acid with positive chitosan. These ionically stabilized layer by layer assemblies can be covalently bound by means of suitable crosslinking chemistry (by means of EDC, etc.). In between, all combinations of polysaccharides are available in a stepless manner by means of common chemical bonding via ester bonds.

In addition, it can be advantageous if the polysaccharide is a chemically modified polysaccharide. In this context, chemically modified means that the polysaccharide has been subjected to artificial modification in the chemical laboratory of a sugar of said polysaccharide, for example a free group, e.g. a hydroxyl, aldehyde or acid group. This allows the range of use to be broadened. For example, inactive groups can be selectively “converted” into active groups, or an undesirable property can be eliminated. For example, treatment with hexamethylene diamine (HMDA) or adipic acid dihydrazide (ADH) or deacetylation can be carried out. HMDA and ADH are both diamide linkers. As ADH shows lower basicity than HMDA, coupling is already possible in the acidic pH range of 4.8. Both linkers thus address coupling chemistry in different pH ranges. Depending on the requirement for bonding of the polysaccharide, different linkers may therefore be required. The polysaccharide can be made hydrolysis-resistant by means of deacetylation.

If the biocompatible material is composed solely of one or several layers, the material for bone implants according to the invention can be applied to solid materials or bodies (basic structure) that are used as bone implants. These bodies may have any possible desired or required three-dimensional shape. Preferably, the entire surface of the material for bone implants according to the invention comprises or consists of the material designed under sub-item (a) above. Suitable materials to which the material according to the invention can be applied can be selected from the ceramic materials, metals, polymers, composite materials or combinations thereof known in the prior art. Examples would be, as metals, titanium/stainless steel; as ceramics, zircon (dioxide), as polymers, poly ether ketone (PEK) and the entire PEK family, but in particular polyether ether ketone (PEEK), polyether ketone ketone (PEKK), polyether ketone ether ketone ketone (PEKEKK); carbon-fiber-reinforced PEEK (CFR-PEEK), PEEK-COMPOSITE, glass-fiber-reinforced polymers, polyethylene (PE), ultra-high-molecular-weight polyethylene (UHMWPE), polyorthoester, polymethyl methacrylate (PMMA), polyethylene terephthalate (PET), polyamide (PA), polylactide (PLA) or polyphenylene sulfone (PPSU). In a preferred embodiment, the material is PEEK. This material is highly similar to mechanical native bone material and is therefore highly suitable as a bone implant material.

Polyether ether ketone (PEEK) having the following structural section

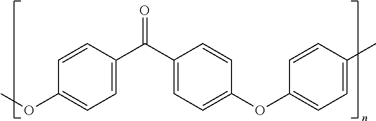

is an extremely widespread thermoplastic high-performance plastic. This semicrystalline plastic is quite popular for applications at higher temperatures due its high solvent resistance, its high melting point of 343° C. and its high glass transition temperature of approx. 143° C.

Since 1998, PEEK has been available on the market in implantable quality. Since then, the market share of PEEK as an implant material has sharply increased. PEEK is used in a variety of applications in medical technology as a bone replacement material and implant, for example as fusion cage for spinal fusion in intravertebral disk injuries. As PEEK is permeable to x-ray radiation and also does not interact with magnetic fields, the placement can be easily monitored after surgery using imaging methods in order to follow the healing process in the affected area. The favorable mechanical properties of PEEK, which highly resemble those of the corticalis (cortical bone), qualify it as a highly suitable material for use as a bone replacement material and in implants.

PEEK is among the materials that behave in almost completely bioinert fashion, i.e. do not undergo any specific interaction with the body. PEEK is neither rejected by the body nor favorably integrated into the bone, and in an ideal case, there is therefore good contact of the bone with the implant. In some cases, tissue encapsulation occurs, which reduces mechanical stability and can lead to loss of the implant. In order to better integrate PEEK into the bone, several methods of achieving bioactivity of the material have been developed, such as e.g. coating with calcium phosphate and also addition of hydroxyapatite particles to the polymer. Other methods of surface modification of PEEK are possible, and are described for example in the following paragraphs.

In order to improve the surface properties of a bone implant so that it is better accepted by the body, it is possible to modify said properties after producing the implant. Surface modifications may be of the physical type, such as e.g. coating with hydroxyapatite (HA) and titanium, or of the chemical type.

In order to chemically modify a plastic substrate such as PEEK, there are in principle two possibilities. One method is that of a direct reaction with small molecules or plasma treatment in order to modify the surface properties and for example introduce linker molecules on which one can carry out further reactions. The concentration of the linker molecules per unit area is naturally extremely low, as the surface of a macroscopic PEEK film is smooth and only the material directly on the surface is accessible. The situation is different in the case of polymer substrates used in solid-phase synthesis. These show good swelling behavior in suitable solvents, allowing linkers to be introduced into the entire volume of the polymer body. In the case of plastics such as PEEK, this behavior is of course undesirable, as the modification is to be limited to the surface in order not to adversely affect volume properties such as hardness or mechanical strength.

Despite this, in order to allow many functional groups to be introduced on the surface, the polymer substrate can be coated with a functional polymer. Depending on the molecular weight of the polymer used, compared to modification with small molecules, this allows many times more functional groups to be introduced.

In this context, a distinction is made between two methods. One speaks of the grafting-from method when a polymer chain is initiated beginning from the surface of the substrate and then grows to an increasing extent. In this variant, the substrate must be capable, for example, of functioning as an initiator radical in radical polymerization, or it must be possible to induce the substrate to function in this manner by means of an activator reagent. In the grafting-from strategy, higher functionalization density is achieved because the polymer chains grow successively and are not applied as a finished polymer. In the opposite approach, the grafting-to method, one proceeds from a finished polymer, which is grafted by means of a suitable mechanism to the surface. A drawback of this method is that in grafting on of finished polymers, adjacent potential bonding sites are strongly blocked by the macromolecules, which does not occur in the grafting-from method. In contrast, the polymer length and molecular weight distribution of the applied polymers can only be controlled in the grafting-to method.

Direct modification with small molecules reduction of the surface carbonyl groups of PEEK is possible by means of reduction using sodium borohydride in dimethyl sulfoxide (DMSO) at 120° C. and coupling of primary amines (cf. below and C. Henneuse; B. Goret; J. Marchand-Brynaert, Polymer 1998, 39, 835-844 and C. Henneuse-Boxus; E. Duliere; J. Marchand-Brynaert, European Polymer Journal 2001, 37, 9-18).

The method is known of coupling different molecules, including gelatin, to the reduced PEEK surface, which made it possible to increase hydrophilicity and achieve higher acceptance of bone-forming cells. The contact angle of the coated substrates also decreased significantly, making it possible to increase the hydrophilicity of the surface and thus the acceptance of bone-forming cells (cf. J. Knaus, Master's Thesis 2013, University of Constance and H. Cölfen; L. F. Tian; J. Knaus, 2016).

For example, surface-induced polymerization can be used as a grafting-from approach. Surface modification by means of small molecules makes it possible to introduce linkers into numerous organic polymers linkers, thus allowing further modifications on the surface. For example, researchers succeed in introducing OH groups on the surface of PEEK by means of wet chemical modification, to which ATRP initiators were then coupled by means of acrylic acid derivatives (cf. B. Yameen; M. Alvarez; O. Azzaroni; U. Jonas; W. Knoll, Langmuir 2009, 25, 6214-6220).

Moreover, UV-induced polymerization (UV: ultraviolet) is also a possibility. Instead of placing a radical starter or other initiators on the surface, it is possible depending on the substrate to produce radicals directly on the surface. This can be achieved for example by using e.g. auxiliary reagents such as benzophenone (BP), benzoyl benzoic acid or other photoinitiators, which on UV excitation abstract hydrogen atoms from suitable polymers and thus produce radicals on the polymer surface that can initiate a chain start. After argon plasma treatment in air, polymers such as PET form hydroxyl and peroxide groups on the surface. These can also serve as radical starters under excitation with UV light. This was also carried out with polyethylene under similar conditions.

It is known that the photoinitiator BP undergoes a photo-pinacol reaction under the action of UV radiation. This results in the formation of a semibenzopinacol radical, which can serve as an initiator in polymerization reactions. By means of photo-induced cleavage, radicals that initiate polymerization can also be generated from the excited molecule. The polymer polyether ether ketone (PEEK) possesses in its polymer backbone BP units, which behave in a similar manner (also see M. Kyomoto; K. Ishihara, Acs. Applied Materials & Interfaces 2009, 1, 537-542). It was demonstrated in 2009 by Kyomoto that under the effect of UV radiation, the surface of untreated PEEK is suitable for initiating radical polymerization of various acrylic acid derivatives. The mechanism involved is a mixed grafting-from and grafting-to mechanism, as growing polymer chains are started on the surface, and growing polymer chains in solution are also terminated on the surface. Based on this study, further groups carried out self-initiating polymerization under UV excitation, using substances such as acrylic acid. The direct detection of ketyl radicals was successfully carried out by Kyomoto in 2013 by in situ ESR spectroscopy.

The covalently bonded matrix typically has a layer thickness of 100 to 150 nanometers (nm), but can also be thicker or thinner. In particular, the covalently bonded matrix can have a layer thickness of 1 nm to 10 micrometers (μm), preferably 10 nm to 1 μm, more preferably 20 nm to 500 nm, more preferably 30 nm to 300 nm, more preferably 50 nm to 200 nm and most preferably 100 to 150 nm. Moreover, the covalently bonded matrix preferably covers the entire surface of the material for bone implants according to the invention.

An interesting variant of the grafting-to method is the capture of growing radical polymer chains by immobilized radical scavengers on the surface of the substrate (also see P. Yang; J. Y. Xie; J. Yuan; L. Zhang; W. N. Liu; W. T. Yang, Journal of Polymer Science, Part A-Polymer Chemistry 2007, 45, 745-755). It was shown by Yang et al. that it is possible to use the polymerization inhibitor hydroquinone in order to apply polymers to a substrate after the grafting-to method.

In this case, a hydroquinone derivative was coupled to the surface, and normal radical polymerization was carried out using a thermal radical initiator. The immobilized hydroquinone quenched the growing polymer chain by homolytic cleavage of the OH bond, causing the chain to break. The long-lasting aryloxy radical is not capable of starting radical polymerization with the available monomers, but can recombine with a growing polymer radical and thus capture the polymer on the surface.

Finally, the material for bone implants according to the invention comprises calcium phosphate embedded in the aforementioned matrix, preferably calcium orthophosphate in all mineral forms, particularly preferably selected from the group consisting of amorphous calcium orthophosphate (ACP), dicalcium phosphate dihydrate (DCPD; Brushite), octacalcium phosphate and hydroxyapatite, also with partial fluoride, chloride or carbonate substitution, wherein ACP, hydroxyapatite and octacalcium phosphate are particularly preferred. Methods for embedding the aforementioned calcium phosphates in a corresponding matrix are described below.

In a preferred embodiment, the polysaccharide is bonded via a linker to the biocompatible material, wherein the linker is selected from a group consisting of: a diamine linker or diamine linkers in combination with a succinic acid linker, a (UV-grafted) polyacrylic acid linker or a photocoupleable linker, in particular an azidoaniline linker. Corresponding linkers are known in the prior art.

Photocoupleable is to be understood as referring to photoreactive or light-induced/light-inducible coupling. Such light-induced coupling has been found to be particularly advantageous, as it is based solely on light and water, thus completely constituting “green chemistry.” In addition, the reaction can be carried out rapidly, efficiently, and in one reaction batch. In addition, no further byproducts are generated, other than the gaseous nitrogen, which can simply escape. This obviates the need for complex purification. Moreover, such an approach has favorable scalability with respect to larger substrates. An example of a procedure would be painting of the implant with subsequent drying, and finally irradiation of every area of the implant. Examples of suitable photocoupleable linkers are azidoanilines, aryl azides or diazirines.

Such light-inducible linkers can be used in combination with a variety of substances or monomers, such as vinyl or acrylic monomers, or polymers (polysaccharides), or a plurality of substances can be radically polymerized with UV light. Examples to be mentioned here include: methacrylic acid, phosphoric acid 2-hydroxyethyl methacrylate ester (as a mixture of monoesters and diesters, with a monoester as a normal monomer or a diester as a linker), 2-hydroxyethyl methacrylate (as a normal monomer or comonomer), ethylene glycol dimethacrylate (as a crosslinker, mixture with other monomers), bis[2-(methacryloyloxy)ethyl]phosphate (as a crosslinker, mixture with other monomers) and 2-(dimethylamino)ethyl methacrylate.

Coupling, e.g. with azidoanilines, can be applied to all polysaccharides that have carboxyl groups and are directly functionalizable analogously to hyaluronic acid, such as e.g. alginic acid, pectin, or carboxymethylcellulose. This advantageously makes a broad range of substances available for use. In this process, analogously to the formation of carboxymethylcellulose, the hydroxyl group present at position 6 of most polysaccharides can be functionalized to form a carboxyl functional group (e.g. chitosan) and is thus available for coupling with azidoanilines.

Methods for the covalent bonding of polysaccharides, for example to PEEK, are described below.

In other embodiments, the material for bone implants according to the invention comprises oxide ceramic materials, titanium, polymer materials or composite materials or consists thereof, wherein the polysaccharide of the covalently bonded matrix in the case of titanium or oxide ceramic materials is bonded via a silane linker. Suitable silane linkers and corresponding methods for the bonding of polysaccharides are known in the prior art.

In a particularly preferred embodiment, the present invention relates to a material for bone implants, wherein:

(a) the biocompatible material is PEEK,
(b) the polysaccharide is alginic acid and
(c) the calcium phosphate embedded in said matrix is hydroxyapatite, in particular crystalline hydroxyapatite.

A further subject matter of the present invention relates to a method for producing a material for bone implants according to the invention comprising the following steps:

a) providing a carrier structure having a surface comprising a biocompatible material,
b) covalent coupling of a matrix comprising at least one polysaccharide to this surface, and
c) mineralizing the matrix with calcium phosphate.

For this subject matter of the present invention, all relevant definitions, advantages and preferred embodiments mentioned above for the material according to the invention for a bone implant apply mutatis mutandis.

Methods for the covalent coupling of a matrix comprising a polysaccharide to a surface according to step (b) of the method according to the invention are not subject to any particular restrictions and are known in the prior art.

In a preferred embodiment, in particular when the surface comprises or consists of PEEK, step (b) of the method according to the invention comprises the following steps in any desired order:

(b1) covalent coupling of a linker molecule selected from the group consisting of a diamine linker or diamine linkers and a succinic acid linker or UV-grafted polyacrylic acid (PAA), or a photocoupleable linker, in particular an azidoaniline linker, to this activated surface, and
(b2) covalent coupling of the polysaccharide with carboxylic acid groups to the diamine linker molecule or of the hexamethylene-diamine-modified polysaccharide to the succinic acid linkers or of the unmodified polysaccharide via ester bonds to the polyacrylic acid linker or the photocoupleable linker, in particular the azidoaniline linker.

In this context, the term “in any desired order” is to be understood as meaning that either coupling of the linker to the activated surface is first carried out, followed by coupling of the product from the activated surface and linker to the polysaccharide, or vice versa, i.e. first the coupling of the linker to the polysaccharide and then the coupling of the product of the linker and polysaccharide to the activated surface.

Methods for the coupling of linker molecules to a correspondingly activated PEEK surface or the activated polymer are also not subject to any particular restrictions.

Advantageously, the covalent coupling of the photocoupleable linker, in particular the azidoaniline linker, to the activated surface takes place at a wavelength in a range of 200 nm to 400 nm, preferably in a range of 200 nm to 300 nm and particularly preferably in a range of 240 nm to 260 nm. According to a preferred implementation of the method, the covalent coupling of the photocoupleable linker, in particular the azidoaniline linker, to the activated surface takes place at a wavelength of 254 nm. This allows a common method to be used in a rapid, reliable, and simple manner.

Preferably, the polysaccharide with its carboxylic acid groups is coupled prior to the light-induced coupling to the photocoupleable linker, in particular the azidoaniline linker.

Preferably, the covalent coupling of the carboxy-functionalized polysaccharide takes place by means of amine and carboxyl group coupling, mediated for example by 1-ethyl-3-(3-dimethylaminopropyl)carbodiimide (EDC), to the photocoupleable linker, in particular the azidoaniline linker. This allows the coupling to be carried out routinely and rapidly using a standard method.

Methods for mineralization of a polysaccharide-containing matrix with calcium phosphates according to step (c) of the method according to the invention are not subject to any particular restrictions. For example, in cases where amorphous calcium phosphate (ACP) is used, these methods include incubation of the surface with a solution comprising calcium chloride, dipotassium hydrogen phosphate and a nucleation inhibitor. This nucleation inhibitor is preferably a non-collagenous protein or protein analog, particularly preferably polyaspartic acid and/or fetuin. In cases where hydroxyapatite is used, these methods include for example incubation of the surface with a solution comprising calcium chloride and dipotassium hydrogen phosphate.

A further subject matter of the present invention relates to a bone implant to which the bone implant material according to the invention is applied.

For this subject matter of the present invention, all relevant definitions, advantages and preferred embodiments mentioned above for the material for bone implants according to the invention apply mutatis mutandis.

A further subject matter of the present invention relates to the use of the bone implant material according to the invention as a bone implant material. For this subject matter of the present invention, all relevant definitions, advantages and preferred embodiments mentioned above for the material for bone implants according to the invention apply mutatis mutandis.

A further subject matter of the present invention relates to the use of the bone implant material according to the invention e.g. for the treatment of bone damage.

For this subject matter of the present invention as well, all relevant definitions, advantages and preferred embodiments mentioned above for the material for bone implants according to the invention apply mutatis mutandis.

A further subject matter of the present invention relates to the use of the bone implant according to the invention e.g. for the treatment of bone damage.

For this subject matter of the present invention as well, all relevant definitions, advantages and preferred embodiments mentioned above for the material for bone implants according to the invention apply mutatis mutandis.

The more closely the implant surface corresponds to the natural bone, the better implants will grow into the human body, and the more stable their bond with the body will be (for reasons such as increased accumulation of somatic cells). This is the object of the present invention. Furthermore, the coating is to be covalently bonded to the surface of the implants. The bone implant materials according to the present invention show higher biocompatibility, better healing into the natural bone and increased mechanical load-bearing capacity.

The aim of the surface modification according to the present invention is to apply bonelike structures by covalent bonding to the surface of bone implant materials containing an organic polysaccharide matrix and the mineral phase of the natural bone. This is intended to support healing of the implant into the bone. These structures contain a matrix of a polysaccharide that is ultimately mineralized with calcium phosphate. The mineralization takes place by means of non-collagenous proteins and their analogs, which function as nucleation inhibitors so that mineralization takes place in a controlled manner and ectopic mineralization is prevented. Examples of such nucleation inhibitors are for example polyaspartic acid or fetuin. The mineralization with octacalcium phosphate or hydroxyapatite takes place by incubation of the polysaccharide matrix in a solution containing calcium ions or phosphate ions. By adding a solution of the respectively complementary phosphate ions or calcium ions in a slow and controlled manner, octacalcium phosphate and/or hydroxyapatite within the polysaccharide matrix can be precipitated. Because of the relatively disordered structure of the polysaccharide, the resulting surface modification has a woven bonelike or callus-like structure. In this manner, during healing of the material, the bone cells could build up further disordered collagen structures around the material or further directly link the material to the bone. These disordered structures could then finally be rebuilt into ordered bone structures during the natural remodeling phase of bone wound healing. However, because of the covalent bonding of the polysaccharides during remodeling, the cells cannot penetrate to the direct surface of the implant material and thus permanently remain in a desired matrix of extracellular proteins. The implant material is thus masked for the cells in order to avoid adverse reactions during healing of implants. As the modifications concern only the surface of the implant materials, material properties are not altered.

The basic chemical reactions can easily be adapted for the modification of different materials. For example, metal oxide surfaces can be covalently bonded by means of established silane chemistry. This also makes the surface coating according to the invention attractive for oxide ceramic materials. As further metals are easily oxidizable on the surface, for example by plasma treatment, common titanium implant materials also become accessible to the surface modification according to the invention by silanes via silane chemistry.

In the past few years, many different materials have been developed for use as bone implants. The biological, chemical, and mechanical requirements for implant materials must be combined in a single material in order to approximate bone properties as closely as possible. The wide variety of approved materials reflects the extensive efforts being made in this field. The plastic polyether ether ketone (PEEK), for example, has highly favorable mechanical properties that are comparable to natural bone. However, their drawbacks, in the form of high hydrophobicity and thus low bioactivity, are obvious, which is why major efforts are under way in order deal with these problems.

A method was developed in which gelatin functionalization of the bone implant plastic PEEK was established. In the present invention, a method has not been developed of covalently binding a network of polysaccharides of plant or bacterial origin, specifically hyaluronic acid and alginic acid derivatives and fully synthetic polymers, to the surface of PEEK in order to avoid the problems connected with an animal-based coating, such as e.g. establishing asepsis and the lengthy approval process due to the potential endotoxin content and the allergenic potential. Some patients decide against certain animal produces for personal reasons, whether for religious or ethical reasons. Vegan or synthetic functionalization can be an attractive alternative for this group of people. In order to approximate the chemical structure of natural bone material as closely as possible, the applied coating can finally be mineralized with calcium phosphate, especially hydroxyapatite.

The description given above of advantageous embodiments of the invention includes numerous individual features that are combined into multiple features in several dependent claims. However, these features can also be considered individually as appropriate and combined into further advantageous combinations, in particular by means of back-references of claims, so that an individual feature of a dependent claim can be combined with one individual, several or all features of another dependent claim. In addition, these features can be combined respectively and in any desired combination both with the method according to the invention and with the device of the invention according to the independent claims. Therefore, process features are also to be seen as representationally formulated as a property of the corresponding device unit, and functional device features are also to be seen as corresponding process features.

The above-described properties, features and advantages of the present invention, as well as the manner in which these are achieved, will be more clearly and unambiguously understandable in connection with the following description of the examples, and the examples given in the following description do not limit the invention to the combination of features given therein, even with respect to functional features. In addition, suitable features of each example can also be explicitly considered in isolation, taken out of an example, inserted in another example in order to complement it, and/or combined with any desired claim.

Other features which are considered as characteristic for the invention are set forth in the appended claims.

Although the invention is illustrated and described herein as embodied in a method for producing a material for a bone implant, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE DRAWING

The FIGURE of the drawings shows correspondence between x-ray powder diffractogram produced with scratched-off deposition on sample plate IS019 and signal of hydroxyapatite from the literature.

### DETAILED DESCRIPTION OF THE INVENTION

The methods mentioned and used in the following text (ATR-IR analysis, scanning electron microscopy, confocal laser scanning microscopy, fluorescence spectrometry, NMR measurements, thermogravimetric analysis (TGA), UV tests, contact angle measurement, nuclear magnetic resonance spectroscopy) were carried out according to the principles and methods known to the person having ordinary skill in the art using known devices.

It is possible by means of wet chemical modification of the PEEK surface to achieve a reduction in the carbonyl groups of the PEEK framework by treating the film with NaBH₄ in DMSO at 120° C.:

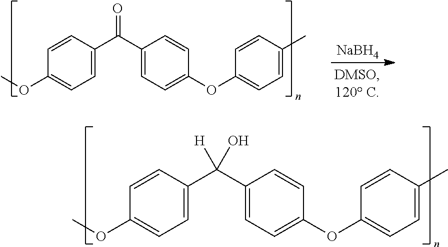

The reaction can be carried out without exclusion of oxygen under stirring in a 500 milliliter (mL) flask. 10 PEEK plates (1 cm² (square centimeter) each) were placed in 20 milliliters (mL) of DMSO and stirred. Heating was carried out to 120° C., and after 20 min, 13 mmol (490 mg) of NaBH₄ was added. The reaction time was 4 h 30 min. The PEEK was washed for 15 min in 20 mL of MeOH, 10 min in 20 ml of H₂O and 35 min in 20 mL of 1 M HCl. After rinsing in EtOH, the PEEK was dried in a vacuum drying oven for 2 h at 40° C. and 50 mbar. The reaction was verified by means of an ATR-IR spectrum (ATR-IR: v 3400 cm⁻¹ (m) (cm⁻¹: wave number), strong attenuation of the 1647 cm⁻¹ (w) C═O stretching vibration, not shown).

The reduced PEEK films can be further converted to the succinic acid ester. The esterification can be carried out by means of succinic anhydride in acetone at room temperature:

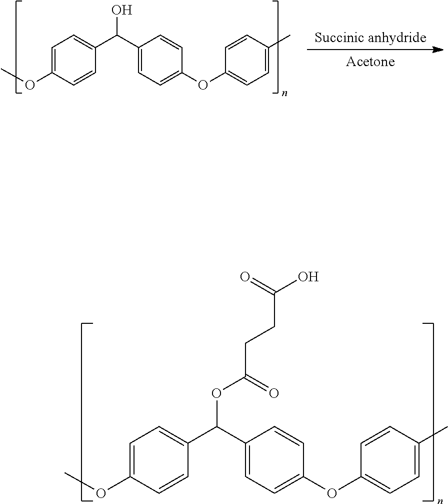

10 PEEK plates (1 cm² each) were placed in 30 ml of acetone and heated to 40° C. 1 gram (g) (10 mmol) of succinic anhydride was added. The reaction time was 5 h 35 min. The plates were washed with 20 mL of acetone, H₂O and ethanol each and dried in a vacuum drying oven for 2 h at 40° C. and 50 mbar. The reaction was verified using an ATR-IR spectrum. ATR-IR: v 3400 cm⁻¹ (m), 2924 cm⁻¹ (w), 2861 cm⁻¹ (m) spa CH₂ stretching vibrations, 1705 (w) COOH stretching vibration (not shown).

Purified PEEK films can be converted to pure diamine (ethylenediamine (EDA) and 1,3-diaminopropane). Imines (Schiff bases) are formed on the PEEK surface. The reaction can be carried out for 3 h under reflux of the diamine and stirring of the mixture:

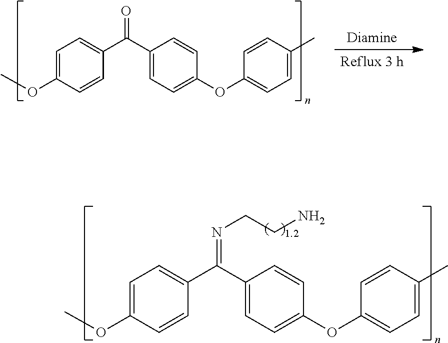

5 PEEK plates (1 cm² each) were placed in 10 mL of ethylenediamine or 1,3-diaminopropane. The reaction mixture was heated under reflux while stirring for 3 h. The reaction mixture was cooled to RT, and the PEEK plates were thoroughly washed with acetone. The modified films were dried in the vacuum drying oven for 2 h at 40° C. and 50 mbar. The reaction was verified using an ATR-IR spectrum ATR-IR: v 2925 cm⁻¹ (m), 2854 cm⁻¹ (m) spa CH₂ stretching vibrations, 1620 cm⁻¹ (w) C═N stretching vibration (not shown).

We were also able to introduce a succinic acid linker into the amine-functionalized PEEK samples: the PEEK films were added to 10 mL of dry 10 millimole (mM) succinic anhydride DMF-solution (DMF: dimethylformamide). After 10 h, the films were carefully washed with MilliQ (ultrapure water) and examined by ATR-IR spectroscopy (not shown). ATR-IR: v 2925 cm⁻¹ (m), 2854 cm⁻¹ (m) spa CH₂ stretching vibrations, 1706 cm⁻¹ (w) C═O stretching vibration.

The modified PEEK films were subjected to contact angle measurement. The modified PEEK films were treated with buffer prior to measurement, rinsed with MilliQ and thoroughly dried. The contact angle measurements can be carried out 5 seconds (s) after drop application. Contact angle measurements with ultrapure water (MilliQ) showed significantly increased hydrophobicity of 66° in ethylenediamine-modified PEEK compared to unmodified PEEK films (84.5°). In the variant modified with 1,3-diaminopropane, the contact angle also decreased, to 70°, as the surface was also more hydrophilic. The increase in hydrophilicity indicates a successful course of the reaction in the conversion of PEEK with diamines.

Untreated PEEK films can be coated with polyacrylic acid using a grafting-from polymerization method (UV-light-induced PEEK-modification):

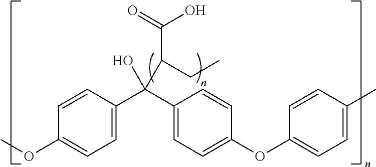

The test specifications used can be carried out in one step. One can work with degassed aqueous solutions of distilled acrylic acid. An OSRAM Vitalux 300 without further filters can be used as a UV light source.

4 PEEK plates (1 cm² each) were placed in a Schlenk flask and degassed in 3 vacuum/nitrogen cycles. The corresponding amount of degassed MilliQ water (4 freeze-pump-thaw cycles) was added. After adding the degassed acrylic acid (30 min nitrogen purging), the flask was irradiated under stirring with the UV lamp from a distance of 15 cm. The reaction time was between 15 min and 75 min. The concentration of the acrylic acid solution was between 5 wt % and 25 wt %. ATR-IR: v 1705 cm⁻¹ (m) COOH stretching vibration.

The respective possible conditions and results are shown in Table 1.

TABLE 1
Reaction batches of UV-grafting with acrylic acid.

| | Acrylic acid | Reaction | Gel |
|---|---|---|---|
| | concentration | time | layer on |
| Item | [wt %] | [min] | surface |
| MG10 | 20 | 75 | yes |
| MG11 | 10 | 70 | yes |
| MG13 | 7.5 | 70 | yes |
| MG19 | 5 | 30 | yes |
| MG20 | 5 | 45 | yes |
| MG12 | 5 | 60 | yes |

The PEEK films grafted with polyacrylic acid films were examined by scanning electron microscopy (SEM) (not shown).

With each batch, an essentially homogeneous layer of polyacrylic acid was formed. With increasing polyacrylic acid concentration and reaction time, an increasing amount of polyacrylic acid was deposited on the surface, essentially in bead form, or the layer thickness increased. The lower the concentration of polyacrylic acid (30 micrometers (μm) for MG10 as compared to 3 μm for MG12), the smaller the beads were.

Samples that were polymerized for 30 min with an acrylic acid content of 5 wt % were used for further surface functionalization. Under these conditions, the coating was still thick enough to be described as a homogeneous coating (not shown), and at the same time it can be assumed with this layer thickness that the mechanical properties of the bulk material are not adversely affected. Because of the thick gel cushion of up to 5 millimeters (mm), the polyacrylic acid layers produced at higher acrylic acid concentrations are not suitable for the target application as bone implant material, as a gel cushion on the surface sharply impairs the quality of the mechanical contact with the surrounding tissue.

Under slight magnification, one can see that the polyacrylic acid has formed linear structures with the beads. The formation of these lines could be due to the drying methods (vacuum oven), but could possibly also be attributable to the hydrophobicity of the PEEK surface. The acrylic acid molecules diffused on the active site have a greater affinity for a growing polyacrylic acid layer than for the hydrophobic PEEK surface. This could also explain the line structures composed of PAA beads. The average bead size under these reaction conditions is 1.7 μm and is thus again approximately half as large as in 60 min polymerization. The coating results were verified by means of ATR-IR spectra (not shown).

The coating produced with 5 wt % of acrylic acid and 30 min UV treatment was found to be particularly suitable. Under these conditions, as a thin layer could be applied to the PEEK, no excessively large gel cushion was deposited.

UV-induced grafting polymerization is thus particularly well suited for the coating of PEEK with polyacrylic acid, as significant amounts of the PAA were detected on the PEEK surface.

It was also possible to carry out polymerization in pure acrylic acid, and as comparison, in pure methyl acrylate, i.e. polymerization in the pure monomer. The results were verified by means of ATR-IR spectra (not shown).

Coupling of 1,4-diaminobutane to the PAA-coated PEEK surface:

The polyacrylic acid layer can be modified by coupling of diamine linkers, so that amide bonds can later be formed with organic acids. Coupling of the diamine species to the carboxyl groups can be carried out using the modern coupling reagent 4-(4,6-dimethoxy-1,3,5-triazin-2-yl)-4-methylmorpholinium chloride (DMT-MM). The activation and coupling can be carried out with DMT-MM at a buffered pH of 9:

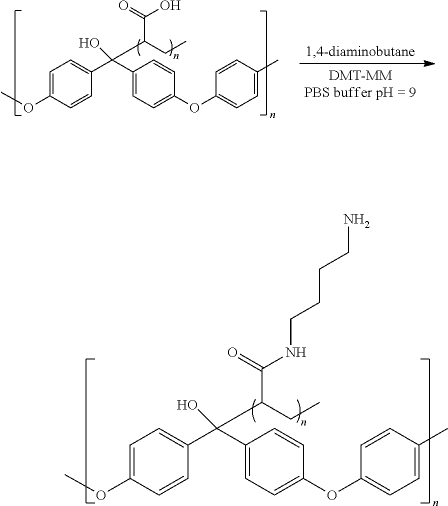

Quantification of the amino groups on the surface of the linker-functionalized PEEK film (substrate) can be carried out in a manner known to the person having ordinary skill in the art by means of the fluorescent dye C-coumarin:

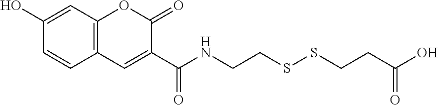 [SMILES: O=C(O)CCSSCCNC(=O)c1cc2ccc(O)cc2oc1=O]

(star=7-hydroxicoumarin fluorophor, Sub=substrate, cf. S. Shiota; S. Yamamoto; A. Shimomura; A. Ojida; T. Nishino; T. Maruyama, Langmuir 2015, 31, 8824-8829):

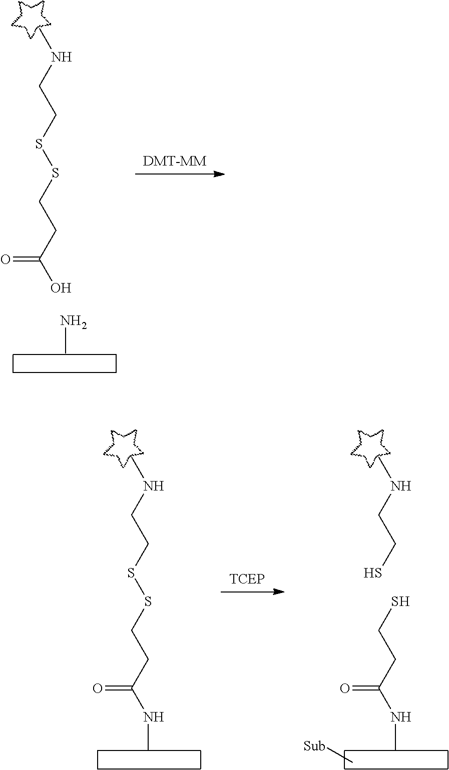

Based on the fluorescence intensity, the concentration of the dye in solution can be determined, thus allowing conclusions to be drawn as to the number of surface amino groups. In the functionalized PEEK samples (with ethylenediamine, 1,3-diaminopropane 4 and tetramethylenediamine (TMDA)), a higher NH₂ density than in a non-functionalized PEEK sample was detected (data not shown).

Synthesis of the modified polysaccharides:

Two strategies can be used for modifying the PEEK films with polysaccharides: introduction of the linker molecule (diamine) on the carboxylate PEEK surface and introduction of the linker on the polymer. In the first case, unmodified polysaccharide is coupled to free amino groups on the substrate in a polymer coupling step, while in the second case, amine-functionalized polysaccharides are anchored to carboxyl groups on the substrate.

In one batch, unmodified hyaluronic acid:

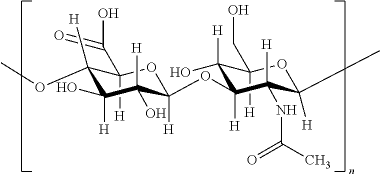

or alginic acid (shown as structural sections of alginic acid with the various poly-G, poly-M and alternating blocks. Depending on the origin of the alginic acid, the ratio of G to M varies):

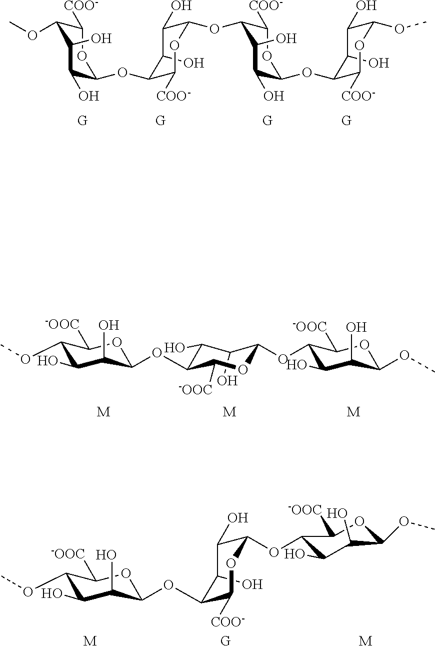

can be functionalized with an amine and then coupled to carboxyl groups on the PEEK surface. Here, it may be necessary to carry out deacetylation before the functionalization.

Deacetylation of hyaluronic acid:

Unmodified hyaluronic acid is composed of a D-glucuronic acid and an N-acetylglucosamine unit. It therefore has one free carboxyl functional group and one acetylated amine functional group per disaccharide monomer. In addition to substitution reactions on the OH groups, two suitable common methods for introducing amine functionalities are functionalization of the carboxyl groups with diamine linkers or the deprotection of the N-acetyl group to form a free amine.

The deacetylation can be carried out in an aqueous hydrazine solution under hydrazine sulfate catalysis:

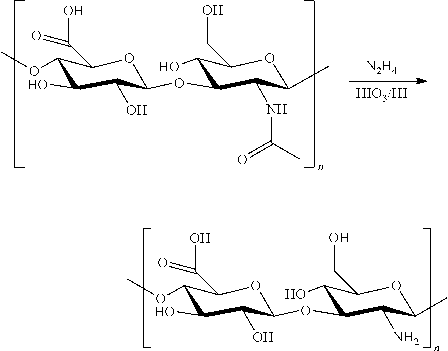

50 mL of hydrazine monohydrate and 0.5 g of hydrazine sulfate were added to 1 g of sodium hyaluronate in order to prepare a 2 percent by weight (wt %) solution based on the polymer. After stirring for 72 h at 55° C., the polymer product was precipitated in cold ethanol, filtered and vacuum-dried (24 h). The residue was taken up in 20 mL of 5% acetic acid. Aqueous iodic acid solution (10 mL, 0.5 M) was added to this solution, wherein the temperature was maintained for 1 h in an ice bath at 4° C. Aqueous hydrogen iodide solution (57%, 3 mL) was added. After 15 min, the violet solution was extracted five times in a separating funnel with 25 mL each of diethyl ether until the aqueous phase was colorless. The pH of the solution was adjusted with a 0.2 M NaOH solution to 7-7.5. The polymer was precipitated in 1 volume equivalent of ethanol, dissolved in H₂O and dialyzed against deionized water. The dialysis water was changed twice daily. After dialysis for 3 days, the solution was freeze-dried and the deacetylated hyaluronic acid was obtained as a product.

The free amino groups could be used as anchor groups for coupling to the carboxyl groups of the PEEK-PAA surface. The polymer was examined by NMR spectroscopy to determine the degree of deacetylation (not shown).

Modification of hyaluronic acid by means of hexamethylene diamine and adipic acid dihydrazide:

The free carboxyl groups of the hyaluronic acid can be suitable for a variety of possible modifications of the polymer. For example, there have been reports in the literature on amidation, esterification or Ugi condensation.

Hexamethylene diamine (NMDA) can be used to synthesize the amidated hyaluronic acid. The coupling of the amine can be carried out via classical EDC/NHS-coupling (1-ethyl-3-(3-dimethylaminopropyl)carbodiimide/N-hydroxysuccinimide coupling) in an aqueous medium. The reaction is divided into an activation phase in slightly acidic buffer solution and a coupling phase in slightly basic buffer solution. The pH of the reaction had to be continuously monitored and readjusted:

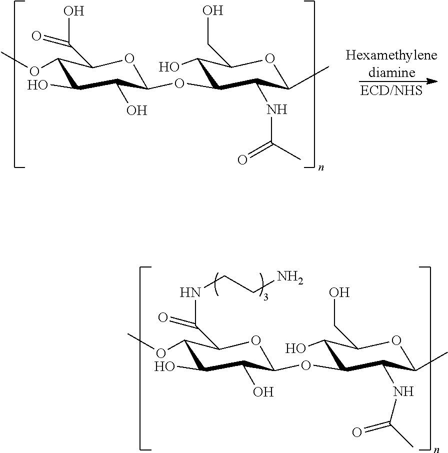

A

$$3 \frac{mg}{mL}$$

aqueous sodium Hyaluronate solution was prepared (500 milligrams (mg) in 167 mL of MilliQ). Based on the number of carboxyl groups in the polymer, 30 eq. of hexamethylene diamine (HMDA, 30 eq., 39.6 mmol, 4.6 g) were added. The pH of the solution was adjusted to 7.5 (0.1 M NaOH, or 0.1 M HCl). EDC (4 eq., 5.28 mmol, 0.9 g) and NHS (4 eq., 5.28 mmol, 0.607 g) were dissolved in 10 mL of water and then added to the reaction solution. The pH of the mixture was maintained at 7.5 by adding 0.1 M NaOH. The reaction solution was stirred overnight. The pH was adjusted to 7, and the polymer was precipitated in ethanol (3 volume equivalents). The polymer was dissolved in MilliQ

$$( 5 \frac{mg}{mL} )$$

and dialyzed for 6 days against FDI water (fully deionized water). The FDI water was changed twice daily. The purified product was freeze-dried for 4 days. The degree of HMDA functionalization was determined by means of ¹H-NMR spectroscopy. 46% HMDA functionalization.

The reaction was carried out with a very large excess of diamine (30-fold, based on the number of carboxyl groups in the polymer) in order to prevent crosslinking of the hyaluronic acid. The successful coupling of the HMDA linker was confirmed by ¹H-NMR spectra, and the successful functionalization of the carboxyl groups by forming amide bonds with the HMDA linkers was confirmed by ATR-IR spectra (not shown).

Modification of hyaluronic acid with adipic acid diazide:

The synthesis specifications were modified for synthesis of the adipic acid dihydrazide (ADH) derivative. As ADH shows lower basicity than HMDA, coupling is already possible in the acidic pH range of 4.8. This made it possible to dispense with the addition of NHS:

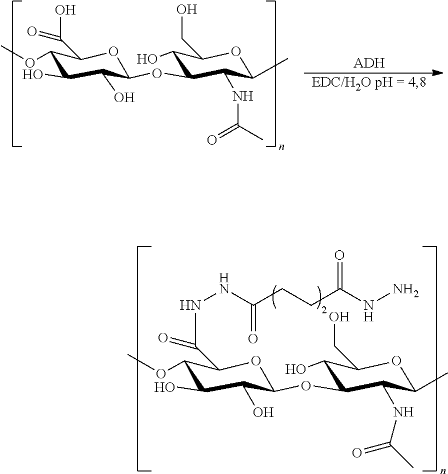

A

$$3 \frac{mg}{mL}$$

sodium hyaluronate solution was produced by dissolving 500 mg sodium hyaluronate in 170 mL of H₂O. Based on the number of carboxyl groups in the polymer, a 40-fold molar excess of adipic acid dihydrazide (ADH, 52.8 mmol, 9.2 g) was added. The ADH was given the time to completely dissolve (15 min). The pH of the reaction mixture was adjusted to 4 using 1 M HCl solution. Ethanol (50 mL, 50 percent by volume (vol %)) was added, and stirring was carried out for 30 min. 4 eq. of EDC-HCl (5.3 mmol, 0.9 g) were added. The pH was maintained for 2 h at approx. 4.8 using 1 M HCl. After 2 h, the reaction was stopped. Neutralization of the solution by means of 1 M NaOH (pH=7). The reaction solution was added to a pre-washed dialysis membrane tube

$$( MWCO = 3500 \frac{g}{mol} )$$

and dialyzed for 9 days. Dialysis was carried out one day against a 100 mM NaCl, followed by alternating dialysis against a 25 vol % ethanol solution one day and against DI water the next. The ethanol/DI water cycle was repeated 4 times. The polymer solution was finally freeze-dried for 3 days. The degree of ADH functionalization was determined by means of ¹H-NMR spectroscopy. 53% ADH functionalization.

The synthesis can be carried out with a large excess of ADH in order to again prevent crosslinking of the hyaluronic acid. In both synthesis processes, the product had to be extensively dialyzed in order to remove the large excess of HMDA or ADH. The successful coupling of the ADH-modified hyaluronic acid was confirmed by ¹H-NMR spectra, and the successful functionalization of the carboxyl groups by forming amide bonds with the ADH was confirmed by ATR-IR spectra (not shown).

HMDA-Modified Alginic Acid:

Analogously to hyaluronic acid, alginic acid can also be functionalized with HMDA. The coupling can be carried out according to a specification for octylamine functionalization:

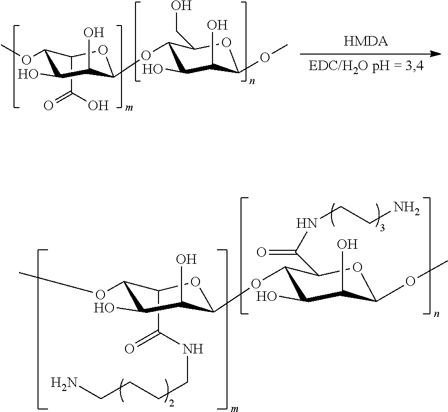

30 mL of 3 wt % aqueous sodium alginate (1 g sodium alginate) was placed in a flask, and the pH was adjusted using 0.1 M HCl to 3.4. The polymer solution was thus diluted to 50 mL (2 wt %). 797.5 mg (4.16 mmol) of EDC-HCl were dissolved in 4 mL of H₂O and added to the polymer solution. The ratio of the EDC to the carboxyl functionalities was thus 0.7. The concentration of the EDC was determined by the molar frequency of the sodium alginate monomers

$$( M = 168.11 \frac{g}{mol} )$$

in the polymer. After a reaction time of 5 min, 10 eq. of hexamethylene diamine (7.05 g) were added. The solution was stirred for 24 h at room temperature. The polymer was precipitated in acetone, dissolved in H₂O after drying, and dialyzed against H₂O. The water was changed twice daily. After dialysis for 9 days, the polymer solution was freeze-dried for 4 days. 938 mg of product was obtained. The degree of HMDA functionalization was determined by means of ¹H-NMR spectroscopy. 31.5% HMDA functionalization.

The synthesis can be carried out with a large excess of HMDA in order to prevent crosslinking of the alginic acid. The product can be extensively dialyzed in order to remove the excess HMDA. The successful coupling of the HMDA linker was confirmed by ¹H-NMR spectra, and the successful functionalization of the carboxyl groups by forming amide bonds with the HMDA linkers was confirmed by ATR-IR spectra (not shown).

Functionalization of the PEEK surface with polysaccharides:

Various strategies can be used for further functionalization of the PEEK surface. On the one hand, various modified polysaccharides can be provided with amine functionalities and in the next step coupled to the carboxyl groups on the PEEK surface. The other approach starts with modification of the carboxyl groups on the surface by diamines, in order to carry out coupling of unmodified polysaccharides in the next step.

Coupling of amine-functionalized polysaccharides:

In order to allow the polysaccharides to be applied to the polymer substrate, a peptide bond should be formed. On the one hand, one can use classical EDC/NHS coupling chemistry. On the other, one can also work with the modern coupling reagent DMT-MM. Classical EDC/NHS coupling can be carried out in two stages. After 20-minute activation of the PEEK film in slightly acidic MES buffer, coupling with the modified polysaccharides can be carried out overnight in slightly basic phosphate buffer:

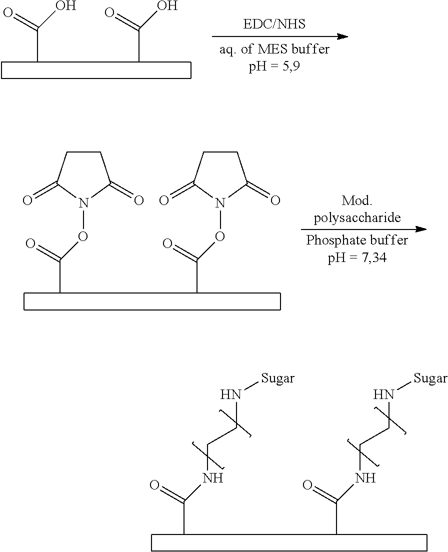

As an alternative coupling reaction, single-stage coupling with the modern coupling reagent 4-(4,6-dimethoxy-1,3,5-triazin-2-yl)-4-methylmorpholinium chloride (DMT-MM) can be selected. The activation mechanism and the subsequent formation of the peptide bond can take place as follows:

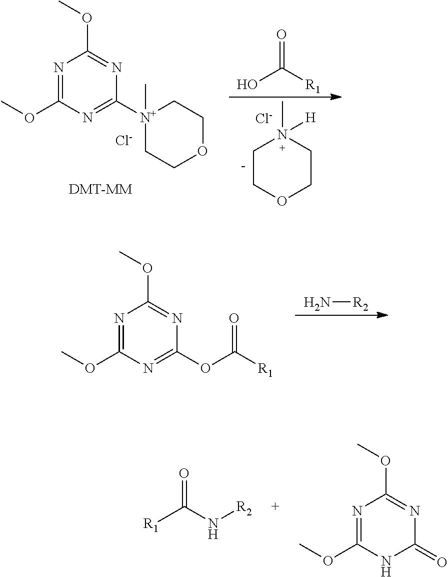

This option is advantageous in that the reaction can be carried out at a constant pH of 9, and the reaction vessel does not have to be changed between activation and coupling. The coupling at a pH of 9 is significantly more rapid than coupling to the NHS ester at a pH of 7.3, because the amines at a pH of 9 are predominantly in the form of free amines, which is important for the nucleophilic attack on the activated carbonyl center. NHS coupling cannot be carried out at such high pH levels, because the NHS ester would be hydrolyzed too quickly in the aqueous solution:

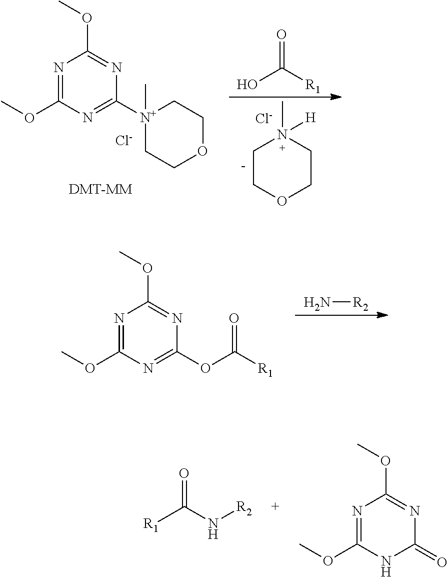

Modification of the imine-functionalized PEEK surface:

The imine-functionalized PEEK surface can be reacted under identical reaction conditions with native hyaluronic acid and alginic acid.

Direct modification of the iminated PEEK surface with native hyaluronic acid. The coupling was carried out by means of DMT-MM in aqueous phosphate buffer at pH=8:

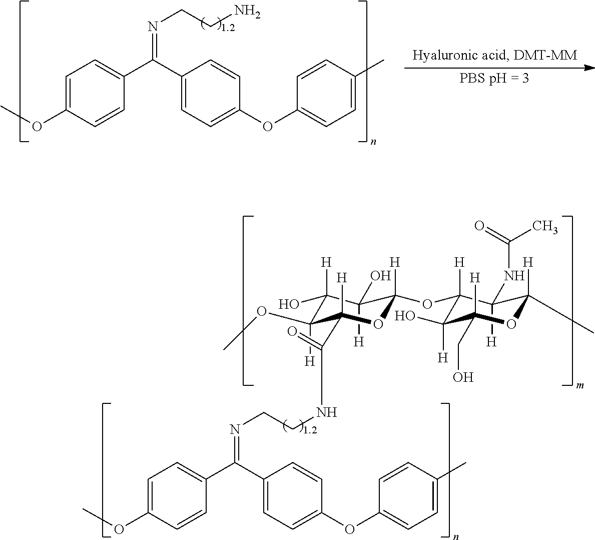

Direct modification of the iminated PEEK surface with native alginic acid. The coupling was carried out by means of DMT-MM in aqueous phosphate buffer at pH=8 (aminated PEEK film was shaken overnight with polysaccharide in PBS buffer (PBS: phosphate-buffered saline) at pH=8 (67 mM) in 1 mM DMT-MM solution. Concentration of hyaluronic acid: 0.1 mg/mL; alginic acid: 0.05 mg/mL. Washing with MilliQ water was carried out three times):

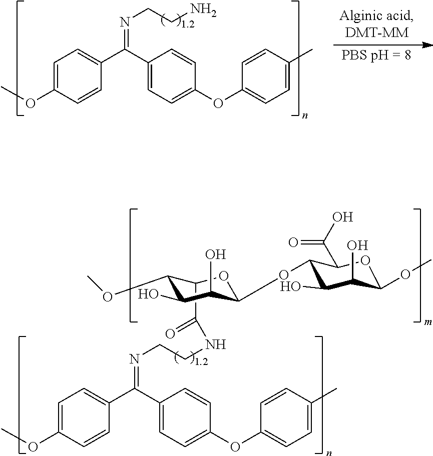

Modification of the PEEK surface coated with polyacrylic acid:

Coating of the PEEK substrates with polyacrylic acid, as described above, was highly successful. The extremely large number of carboxyl groups that were introduced onto the PEEK surface in this manner served as a point of departure for further functionalization with different polysaccharide derivatives. Coupling reactions were thus carried out with ADH-hyaluronic acid, HMDA-hyaluronic acid, deacetylated hyaluronic acid and HMDA-alginic acid.

Modification of the PEEK surface coated with polyacrylic acid with ADH-hyaluronic acid. The coupling was carried out by means of DMT-MM in aqueous phosphate buffer at pH=8:

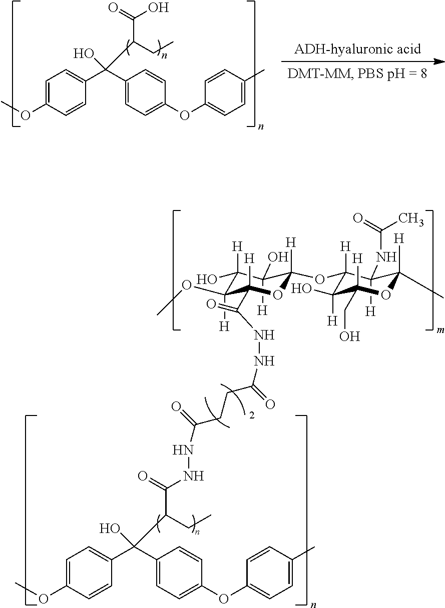

The reaction was carried out under the same conditions as the previous couplings.

Modification of the PEEK surface coated with polyacrylic acid with HMDA-hyaluronic acid. The coupling was carried out by means of DMT-MM in aqueous phosphate buffer at pH=8:

Modification of the PEEK surface coated with polyacrylic acid with HMDA-alginic

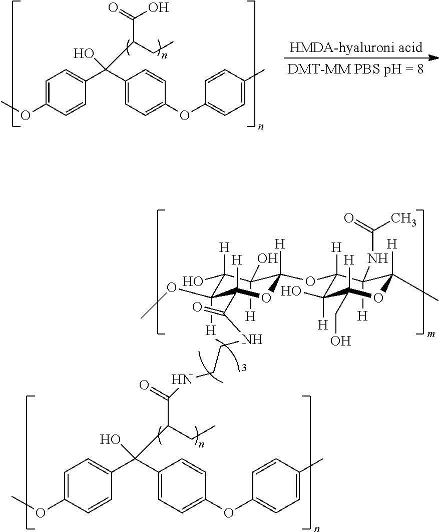

acid. The coupling was carried out by means of DMT-MM in aqueous phosphate buffer at pH=8:

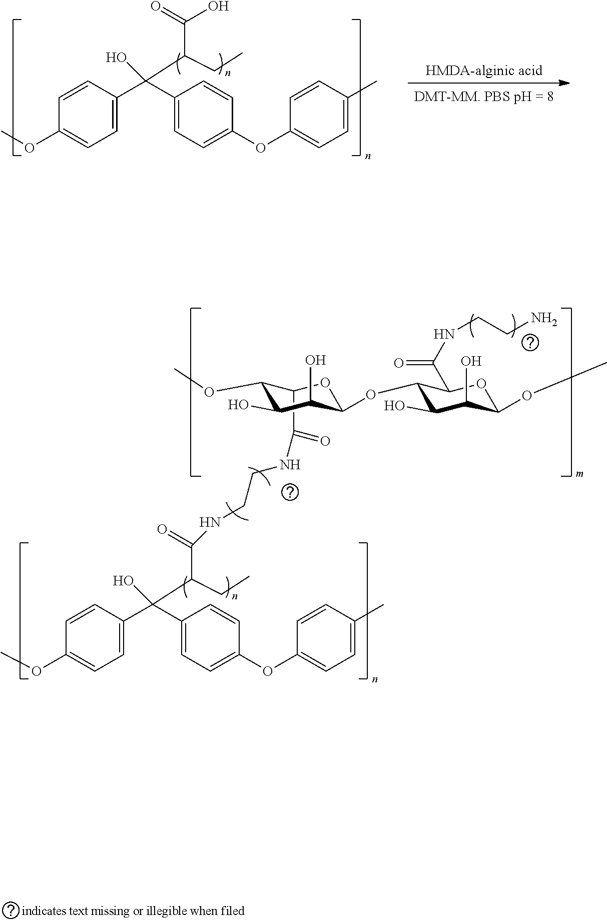

Modification of the PEEK surface coated with polyacrylic acid with deacetylated hyaluronic acid. The coupling was carried out by means of DMT-MM in aqueous phosphate buffer at pH=8:

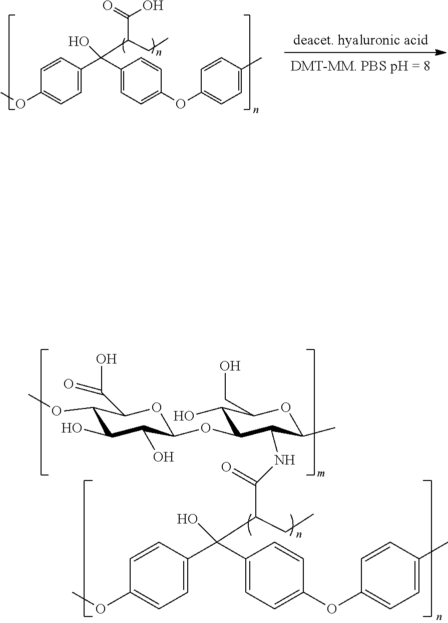

Overall, significantly more coupling tests were carried out on the PEEK substrate. An overview of the reactions is shown in Table 2. The couplings marked with an X were carried out. All reactions were carried out under the same basic conditions using DMT-MM as a coupling reagent.

TABLE 2
Overview of polysaccharide couplings to PEEK
substrates.

| | | | Deac.- | ADH- | HMDA- | HMDA- |
|---|---|---|---|---|---|---|
| | hya | Alg | hya | hya | hya | Alg |
| PEEK-imine | X | X | | | | |
| PEEK-imine- | | | X | X | X | X |
| COOH | | | | | | |
| PEEK-PAA | | | X | X | X | X |
| PEEK-PAA- | X | X | | | | |
| amine | | | | | | |

The reaction diagrams for the individual reactions are as follows: successful functionalization was confirmed by ATR-IR spectra (not shown).

Direct modification of the iminated PEEK surface with native alginic acid. The coupling was carried out by means of DMT-MM in aqueous phosphate buffer at pH=8:

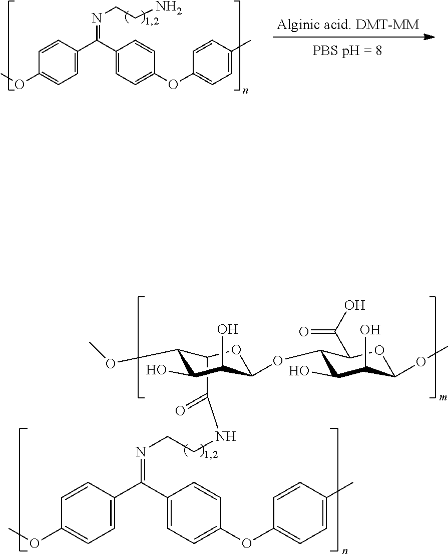

Modification of the iminated and carboxylated PEEK surface with ADH-hyaluronic acid. The coupling was carried out by means of DMT-MM in aqueous phosphate buffer at pH=8:

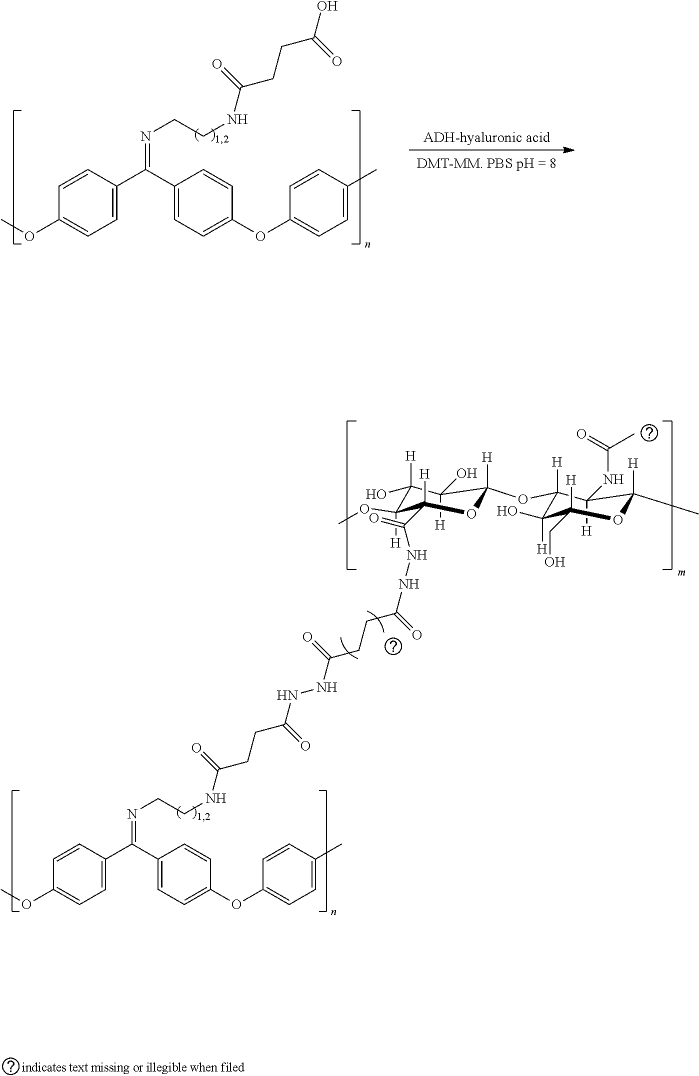

Modification of the iminated and carboxylated PEEK surface with HMDA-hyaluronic acid. The coupling was carried out by means of DMT-MM in aqueous phosphate buffer at pH=8:

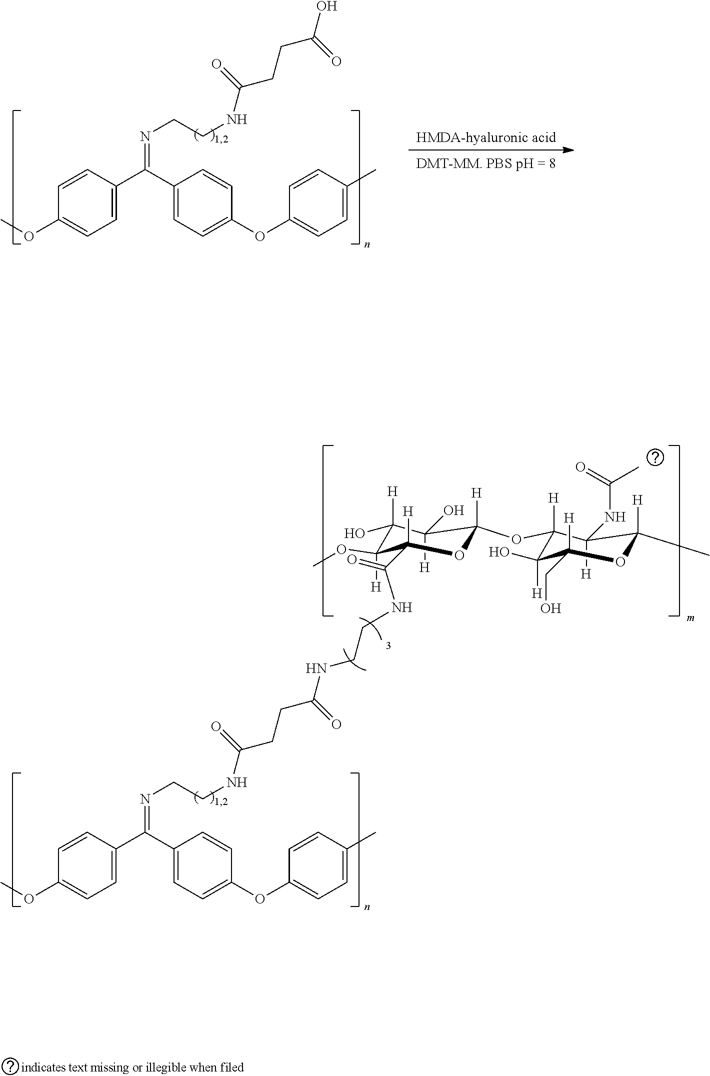

Modification of the iminated and carboxylated (succinic acid) PEEK surface with HMDA-alginic acid. The coupling was carried out by means of DMT-MM in aqueous phosphate buffer at pH=8:

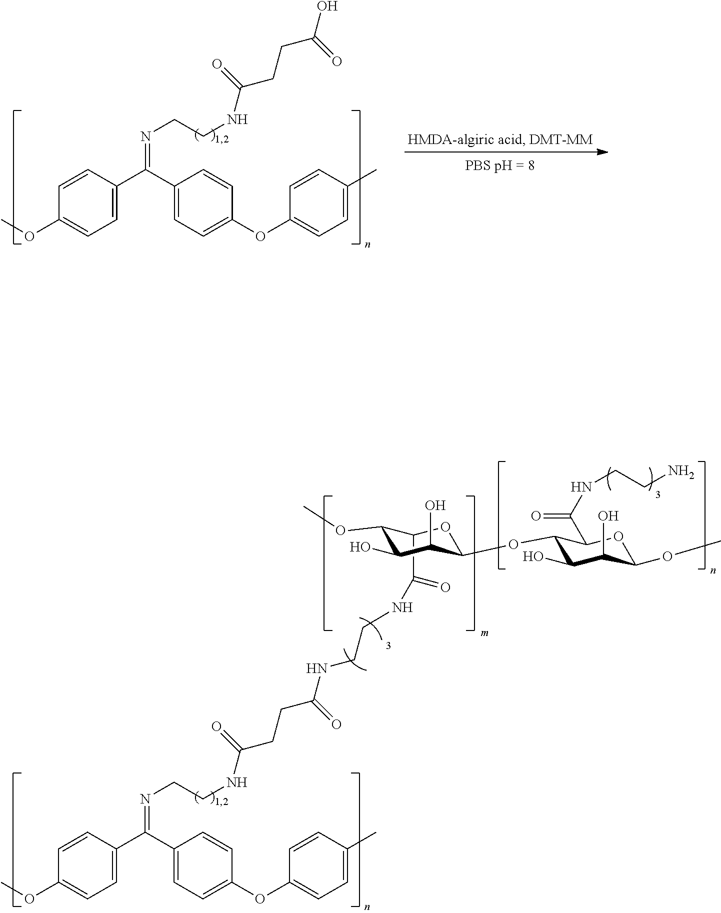

Modification of the iminated and carboxylated PEEK surface with deacetylated hyaluronic acid. The coupling was carried out by means of DMT-MM in aqueous phosphate buffer at pH=8:

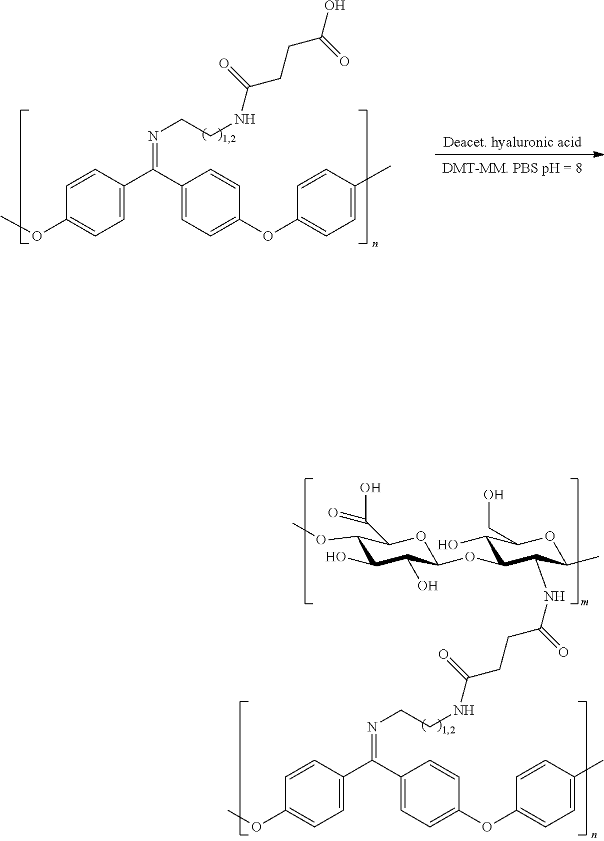

Modification of the PEEK surface coated with polyacrylic acid with ADH-hyaluronic acid. The coupling was carried out by means of DMT-MM in aqueous phosphate buffer at pH=8:

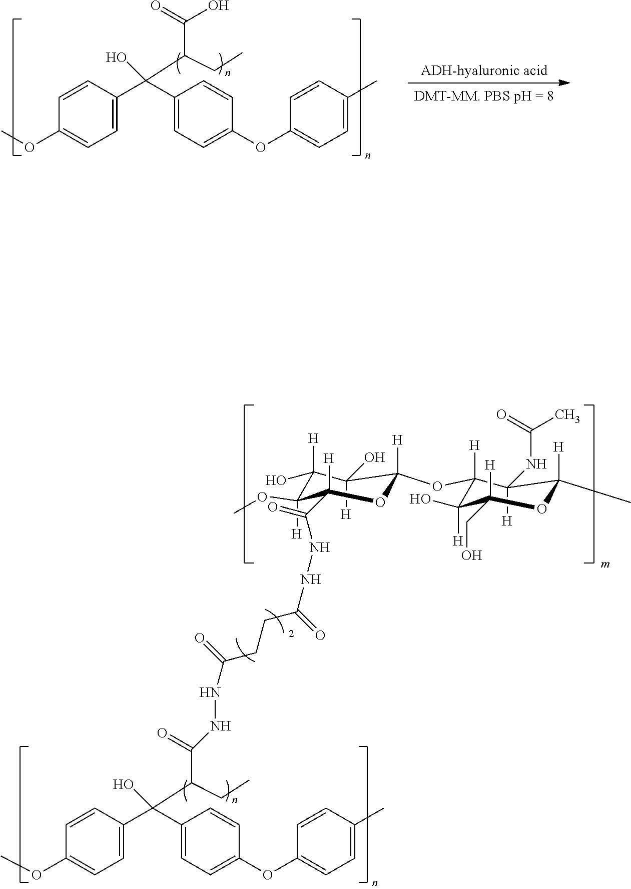

Modification of the PEEK surface coated with polyacrylic acid with HMDA-hyaluronic acid. The coupling was carried out by means of DMT-MM in aqueous phosphate buffer at pH=8:

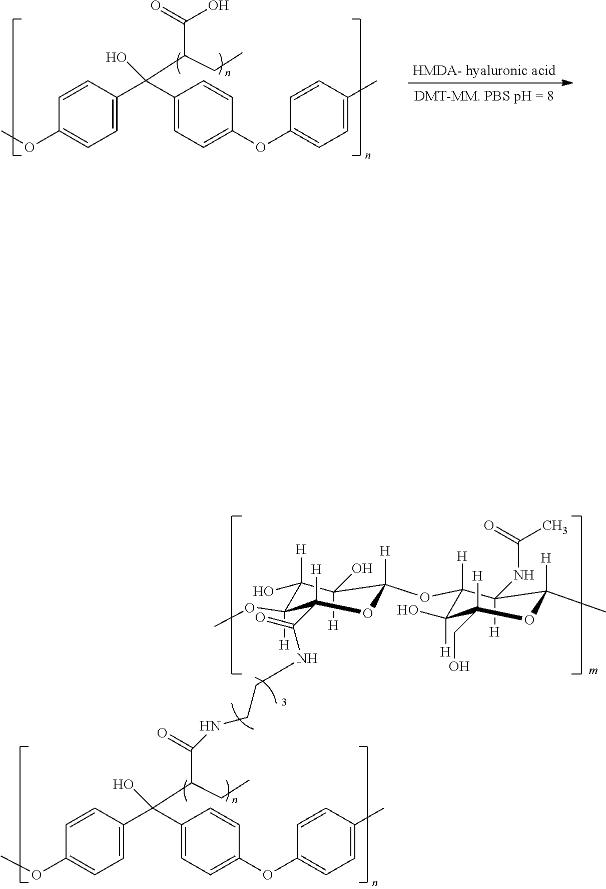

Modification of the PEEK surface coated with polyacrylic acid with HMDA-alginic acid. The coupling was carried out by means of DMT-MM in aqueous phosphate buffer at pH=8:

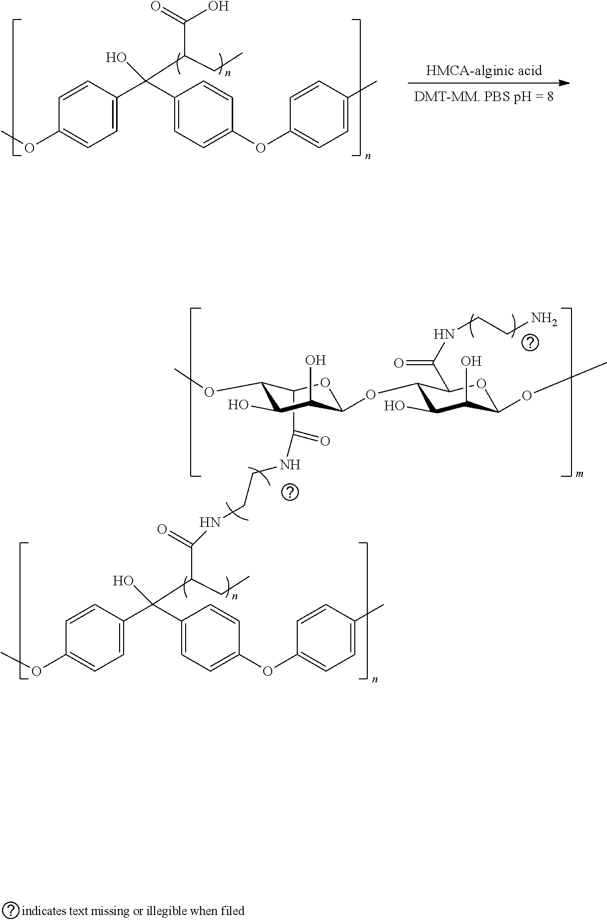

Modification of the PEEK surface coated with polyacrylic acid with deacetylated hyaluronic acid. The coupling was carried out by means of DMT-MM in aqueous phosphate buffer at pH=8:

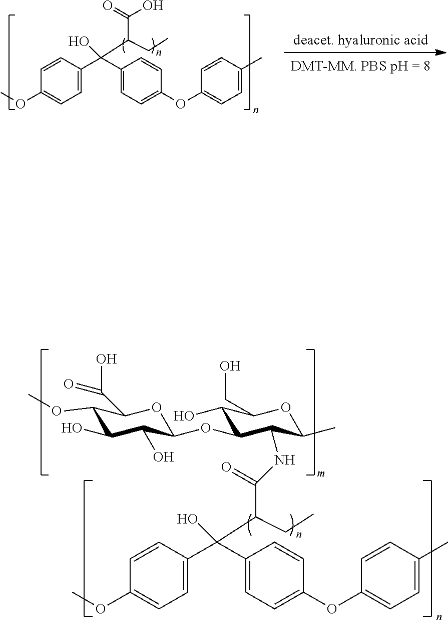

Modification of the PEEK surface coated with polyacrylic acid and then treated with tetramethylene diamine with native hyaluronic acid. The coupling was carried out by means of DMT-MM in aqueous phosphate buffer at pH=8:

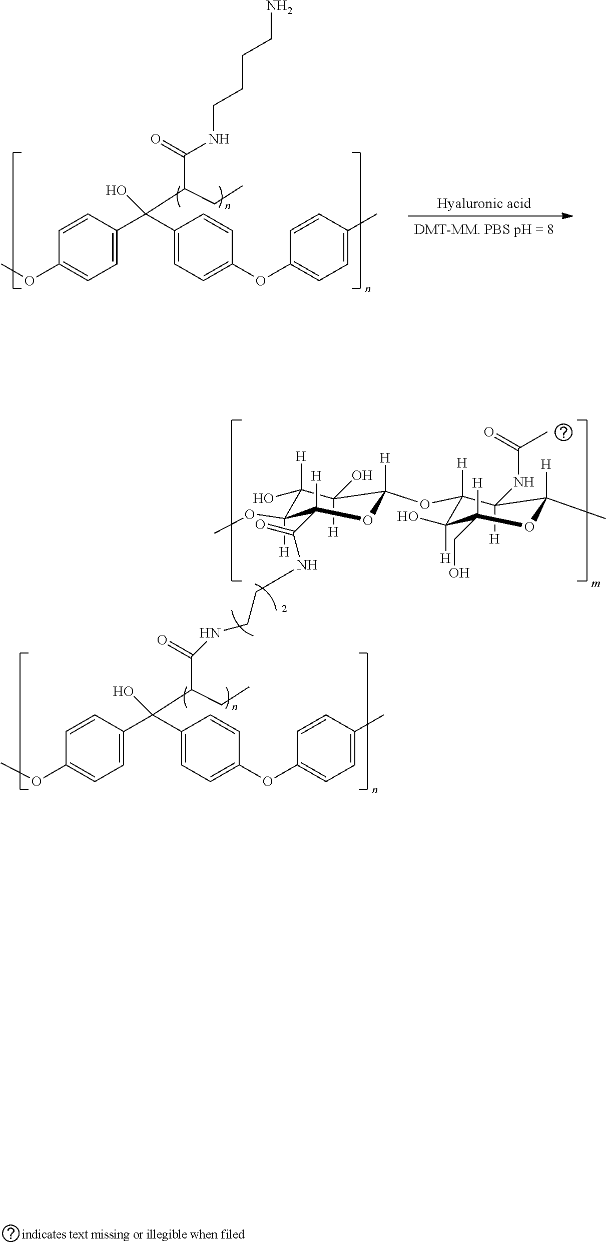

Modification of the PEEK surface coated with polyacrylic acid and then treated with tetramethylene diamine with native alginic acid. The coupling was carried out by means of DMT-MM in aqueous phosphate buffer at pH=8:

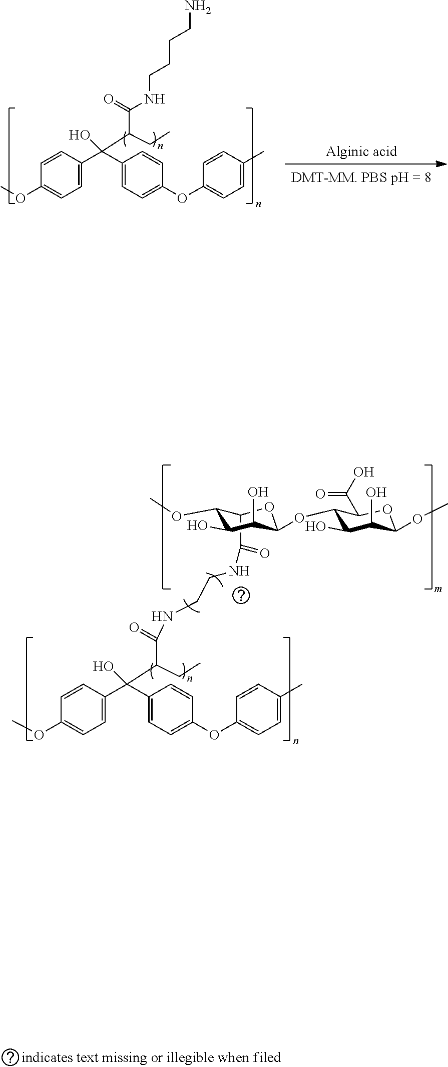

Modification of the iminated PEEK surface with coupled succinic acid with native alginic acid. The coupling was carried out by means of DMT-MM in aqueous phosphate buffer at pH=8:

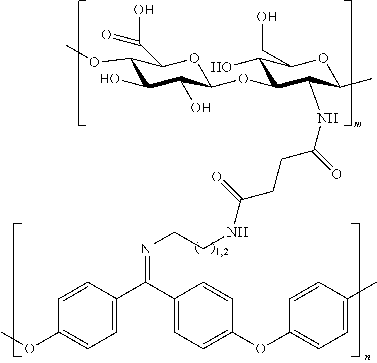

Mineralization tests with hydroxyapatite:

In addition, three different batches for the mineralization of hydroxyapatite per PEEK-PAA sample were tested. First, two tests were carried out with calcium prestructuring and one with phosphate prestructuring.

Here, the following Ca prestructuring would be possible:

An established synthesis route for hydroxyapatite can be modified and used for the mineralization of PEEK-PA films. The PEEK-PA film was placed in 0.3 M calcium chloride solution at a buffered pH of 9 and then stirred for 30 min. A disodium hydrogen phosphate solution, also at a buffered pH of 9, was now added at a rate of

$$3 \frac{mL}{min} .$$

After addition was completed, the mixture was stirred overnight.

Ca prestructuring and phosphate prestructuring were also carried out:

Simpler variants of the mineralization can also be carried out. PEEK-PAA samples were placed for 72 h in diammonium hydrogen phosphate (phosphate prestructuring, 6 mL vial with 1 M aqueous (NH₄)₂HPO₄ solution) or calcium nitrate Cal (6 mL vial with 1 M aqueous Ca(NO₃)₂ solution). After this rest time, each of the samples was placed in the other solution respectively (0.6 M (NH₄)₂HPO₄ or 0.6 M Ca(NO₃)₂ solution) and left therein for one week in order to also allow the counterions to diffuse into the gel.

Photoinducible Coupling

According to a further example, azido-functionalized hyaluronic acid was bonded by light-induced coupling to a PEEK surface. The reaction sequence of the light-induced coupling of azidoanilines with hyaluronic acid to PEEK could be as follows:

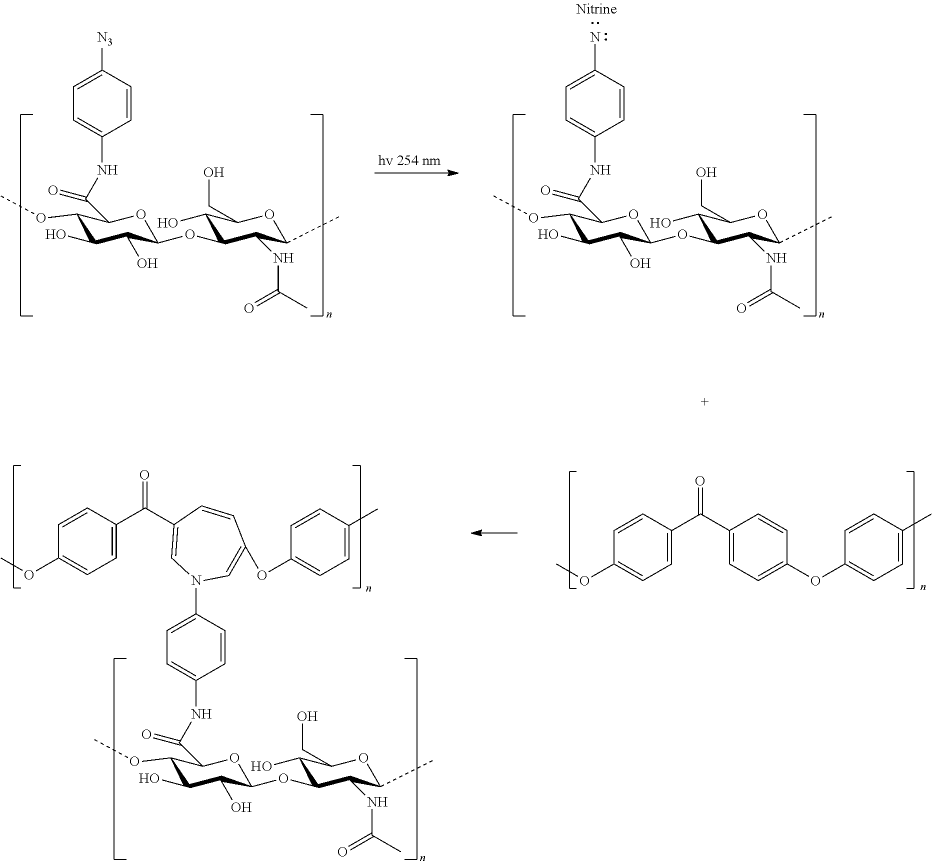

Azidoaniline groups were thus coupled to the carboxy groups of polysaccharides such as e.g. alginic or hyaluronic acid (see below). The coupling product of polysaccharides and azidoaniline linkers was then bonded with light to the PEEK surfaces.

Coupling of the azidoaniline linkers to the polysaccharides takes place in a first step by means of standard EDC-mediated amine and carboxyl group coupling to the carboxyl-functionalized polymer. Large polymers such as high-molecular-weight hyaluronic acid form strong secondary structures (such as helix structures, etc.) that also cause high viscosity. This makes the diffusion of reagents to the functional groups poor and hinders accessibility. For this reason, the hyaluronic acid can be pre-treated if necessary. A suitable means for this would be for example a cleaved hyaluronic acid, such as an enzymatically cleaved or ultrasound-cleaved hyaluronic acid. In enzymatic cleavage, for example with hyaluronidase, the hyaluronic acid is cleaved into fragments of approx. 15 kilodaltons (kD), and in ultrasound treatment into fragments of approx. 300 kD.

Production of an azido-functionalized hyaluronic acid could be carried out according to Eychenne, Romain, et al. “Rhenium Complexes Based on an N20 Tridentate Click Scaffold: From Synthesis, Structural and Theoretical Characterization to a Radiolabeling Study.” European Journal of Inorganic Chemistry 2017.1 (2017): 69-81), or commercially obtained hyaluronic acid could be used.

Synthesis of photoreactive hyaluronic acid (hya-N3) (cf. Chen, Guoping, et al. “Photoimmobilization of sulfated hyaluronic acid for antithrombogenicity.” Bioconjugate Chemistry 8.5 (1997): 730-734.):

Material: 100 mg (1 equivalent) of the (cleaved) hyaluronic acid; 45 mg (1 equivalent) of 4-azidoaniline; 58.2 mg (1.15 equivalents) of EDC-CI (1-ethyl-3-(3-dimethylaminopropyl)carbodiimide HCl);

dissolve all three reactants in 110 mL of MilliQ water;
adjust pH to 7;
stir overnight away from light;
purify by dialysis unit UV signal in dialysis water is no longer detectable at 255 nm;
and
freeze-dry.

Coating of polyether ether ketone (PEEK) with azido-functionalized hyaluronic acids:

wash PEEK substrate with ethyl acetate and acetone respectively for approx. 1 min;
prepare solution of azido-functionalized hyaluronic acids (1 mg/mL) (enzymaticallycleaved azido-functionalized hyaluronic acid “hya-enz-N3” and ultrasound-cleaved azido-functionalized hyaluronic acid “hya-US-N3”) in MilliQ-water and 3 mL vial with snap-on cap by mixing and shaking;
apply 50 μL of the respective solution to washed PEEK substrates with a pipette;
dry overnight in the air (cover the dabbed PEEK films, for example with a 96-well plate, in order to protect the respective substrate type from dust deposits. A snap-on cap placed on the cover provides the necessary ventilation. Also cover with aluminum foil for protection from light.).

Observation 1: A spot of a drop-shaped material deposit is clearly visible after drying (not shown):

spread out the PEEK plates with dried azido-functionalized hyaluronic acid on a cloth; and
irradiate with short-wave UV (254 nm) for 100 min at a distance of approx. 1 cm.

Observation 2: On both substrate types (hya-Enz-N3; hya-US-N3), one can see a round (drop-shaped), brownish deposit that appears darker than the deposit after the drying process (cf. Observation 1). The substrates with hya-US-N3 are somewhat darker colored than the substrates with hya-Enz-N3.

Wash PEEK substrates with hyaluronic acid derivatives after drying and exposure to light for 24 h on a shaker table in MilliQ and a 50 mL Falcon tube.

Dry washed PEEK substrates for 24 h in a Falcon tube with a perforated parafilm cover in the vacuum oven.

Observation 3: The spot of the material deposit is still clearly visible on the washed, irradiated PEEK film substrate treated with a hyaluronic acid derivative. On a similarly washed non-irradiated film, in contrast (negative sample), no spot can be seen. Therefore, material was successfully coupled to the PEEK substrate in a wash-resistant manner. More for hya-US-N3 than for hya-Enz-N3) (not shown).

After this, mineralization tests were carried out with the PEEK substrates coated with hyaluronic acid derivatives produced as described above.

Mineralization Solution:

40.5 g of NaCl; 1.8475 g of CaCl₂); 0.735 g of Na₂HPO₄; 8.875 g of HCl (conc.); 500 mL of MilliQ

Preparation: prepare MilliQ water, then dissolve salts therein and add HCl (weighed out in a syringe). Store in a laboratory flask until use.

Procedure:

Prepare dilution of the mineralization solution depending on the desired pH range

a) pH 7: mineralization solution to MilliQ water in a ratio of 2:8
b) pH 8: mineralization solution to MilliQ water in a ratio of 1:9
c) pH 9: mineralization solution to MilliQ water in a ratio of 1:18
Adjust pH immediately before use with 1 M Trizma® base (Sigma), as this “activates” the solution and initiates the mineralization.

Then immediately add 25 mL of activated mineralization solution to the substrates to be mineralized to 30 mL vials with snap-on caps (1 substrate per vial). Ensure that the coated side faces upward and the plate does not float on the solution, but is fully immersed.

Then place the room temperature samples on a shaking table. Place the samples at elevated temperatures (approx. 37° C.) in a corresponding water bath (raised, so that they are immersed but not completely under water).

After the desired period of time, remove the substrates, wash them in 10 mL of MilliQ in a 15 mL Falcon tube on the shaking table for 30 min, and then dry overnight in a vacuum drying oven.

After this, conduct analysis by scanning electron microscopy (SEM) for surface structures, energy-dispersive x-ray analysis (EDX) for elemental composition, and then x-ray diffraction (XRD) for mineral phase (partially not shown).

Different test batches were carried out (cf. Table 3). PEEK was used as a substrate in all cases, either enzymaticallycleaved azido-functionalized hyaluronic acid (abbreviated as Enz) or ultrasound-cleaved azido-functionalized hyaluronic acid (abbreviated as US) was used as a coating, incubation was carried out at a pH of 7, 8 or 9 (see above for dilutions), and coupling was carried out in all batches by means of UV light. In addition, mineralization blank tests were conducted at all three pH levels and at both temperatures (RT/37° C.) without the presence of a substrate, with no mineralization being detected in any cases (not shown).

TABLE 3
Mineralization test batches

| P | B | pH | T | A | E | T |
|---|---|---|---|---|---|---|
| IS005 | Enz | 7 | 37 | d1, 15:10 | d2, 14:30 | 23 h, 20 min, 1 d |
| IS006 | Enz | 7 | 37 | d1, 15:10 | d5, 13:30 | 118 h, 20 min, 5 d |
| IS008 | Enz | 7 | RT | d1, 15:10 | d2, 14:30 | 23 h, 20 min, 1 d |
| IS009 | Enz | 7 | RT | d1, 15:10 | d5, 13:30 | 118 h, 20 min, 5 d |
| IS019 | Enz | 8 | 37 | d1, 13:45 | d2, 14:15 | 24 h, 30 min, 1 d |
| IS020 | Enz | 8 | 37 | d1, 13:45 | d5, 12:40 | 118 h, 55 min, 5 d |
| IS022 | Enz | 8 | RT | d1, 13:45 | d2, 14:15 | 24 h, 30 min, 1 d |
| IS023 | Enz | 8 | RT | d1, 13:45 | d5, 12:40 | 118 h, 55 min, 5 d |
| IS033 | Enz | 9 | 37 | d1, 14:05 | d2, 13:20 | 2 3 h, 15 min, 1 d |
| IS036 | Enz | 9 | RT | d1, 14:05 | d2, 13:20 | 23 h, 15 min, 1 d |
| IS011 | US | 7 | 37 | d1, 15:10 | d2, 14:30 | 23 h, 20 min, 1 d |
| IS012 | US | 7 | 37 | d1, 15:10 | d5, 13:30 | 118 h, 20 min, 5 d |
| IS014 | US | 7 | RT | d1, 15:10 | d2, 14:30 | 23 h, 20 min, 1 d |
| IS015 | US | 7 | RT | d1, 15:10 | d5, 13:30 | 118 h, 20 min, 5 d |
| IS025 | US | 8 | 37 | d1, 13:45 | d2, 14:15 | 24 h, 30 min, 1 d |
| IS026 | US | 8 | 37 | d1, 13:45 | d5, 12:40 | 118 h, 55 min, 5 d |
| IS028 | US | 8 | RT | d1, 13:45 | d2, 14:15 | 24 h, 30 min, 1 d |
| IS029 | US | 8 | RT | d1, 13:45 | d5, 12:40 | 118 h, 55 min, 5 d |
| IS039 | US | 9 | 37 | d1, 14:05 | d2, 13:20 | 23 h, 15 min, 1 d |
| IS042 | US | 9 | RT | d1, 14:05 | d2, 13:20 | 23 h, 15 min, 1 d |
| IS043 | US | 9 | RT | d1,14:05 | d4, 13:00 | 94 h, 55 min, 4 d |

Legend: P = name of sample, B = coating, pH = pH value, T = temperature in [°C.] (RT = room temperature), A = beginning of incubation (d = day, XX:YY = time), E = end of incubation, t = time in hours [h] and minutes [min], (corresponds to X) day(s) [d].

Observation 4:

In samples IS005, 006, 008, 009, 011, 012, 014, 015, 022, 028, 036, 042 and 043, the hyaluronic acid ring or spot is recognizable on SEM examination. This finding is consistent with signals in the EDX analysis of carbon and oxygen. However, no wash-resistant film or deposit can be seen on the hyaluronic acid coating, nor is any calcium or phosphorus detectable in the EDX analysis that would indicate mineralization.

In samples IS019, 020, 023, 025, 026, 029, 033 and 039, on the other hand, a wash-resistant film or deposits can be seen in the areas of the hyaluronic acid ring or spot. On EDX analysis, in addition to carbon and oxygen, calcium and phosphorus are also observed, which indicates mineralization.

In the following, results are presented by way of example for the samples IS019 (enzymatically cleaved hyaluronic acid coating, pH 8, 37° C., 1 day embedding time) and IS025 (ultrasound-cleaved hyaluronic acid coating, pH 8, 37° C., 1 day embedding time).

In the SEM analyses, deposits are clearly visible on all of the plates. The deposits are not homogeneously, but irregularly distributed. Many areas are covered by a filmlike layer, and other areas show spongelike bead material accumulations. In IS019, the hyaluronic acid coating is a ring (as was the case for all previous samples with enzymatically cleaved hyaluronic acid) and is not completely but partially covered with the deposited material. The agglomerations show no preference for the hyaluronic acid coating or PEEK, but appear to be distributed equally heterogeneously at all sites (not shown). In IS025, in contrast, the hyaluronic acid coating is a filled spot (as was the case for all previous samples with hyaluronic acid cleaved by means of ultrasound) and is covered with an extremely thick layer of the deposited material. There are signs of preferential mineralization of the hyaluronic acid coating and weaker signs of covering of the uncoated PEEK areas (not shown).

EDX analysis of the respective foamlike beaded deposits clearly shows the presence of phosphorus and calcium in addition to carbon and oxygen. The content percentages for IS019 are oxygen (O) 44.6%, carbon (C) 21.8%, calcium (Ca) 20.5% and phosphorus (P) 13.1% and those for IS025 are oxygen (O) 45.2%, carbon (C) 40.9%, calcium (Ca) 9.0% and phosphorus (P) 4.9%. Carbon is attributable to PEEK, the hyaluronic acid and the carbon adhesive tape with which the sample was attached to the sample carrier for the test. Calcium, phosphorus and oxygen indicate the possible presence of calcium phosphate compounds. Mapping was carried out, with the following results (not shown): the calcium and phosphorus signal distribution is consistent with the beadlike material deposits. Carbon is primarily located at sites where the substrate or the hyaluronic acid coating is not covered by the beadlike material. Oxygen is relatively regularly distributed, but particularly at sites where the beadlike material was deposited (and more strongly at sites where the hyaluronic acid layer is present, as said layer contains more oxygen than PEEK).

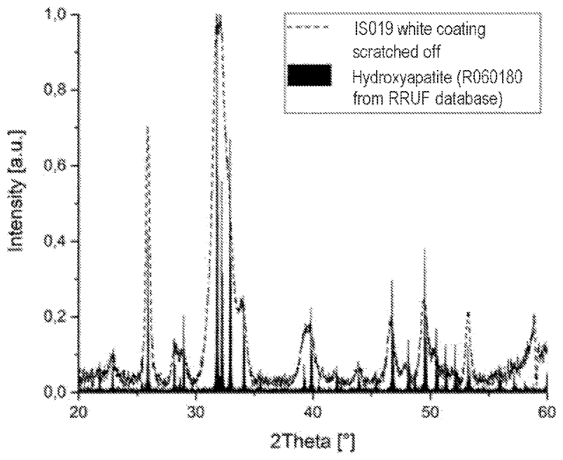

Initial measurements from XRD analysis of the IS0019 sample indicate calcium metaphosphate. However, the signal of the coating is very difficult to read out, because PEEK is polycrystalline and thus emits extremely strong, sharp signals that mask all other signals, and in addition, the hyaluronic acid is only minimally visible. Accordingly, parts of the white deposit were scratched off so that they could be measured individually, i.e. without substrate signals. The x-ray powder diffractogram of the scratched-off deposit is shown in FIG. 1 (dashed graph). In addition, the signal of hydroxyapatite (R060180 from the RRUFF database) was included in the diagram (black graph). It can be seen that there is a high degree of agreement between the signal of the sample IS019 and the literature signal of hydroxyapatite. It can therefore be assumed that in mineralization, hydroxyapatite is produced on the hyaluronic acid coating of PEEK.

The following conclusions can be drawn from the tests:

With respect to the effect of pH, the general trend appears to be that the higher the pH of the mineralization solution, the more rapidly deposition occurs on the substrates. The optical impression currently confirms this for all of the mineralization tests observed so far from pH 7 to pH 9.

A higher temperature (here: 37° C. in the water bath) compared to room temperature (approx. 21° C.) appears to promote the deposition of material on the substrates. It was possible to observe this macroscopically in all previous mineralization tests.

In addition, the deposited materials appear to be rather coarse at RT, while a temperature of 37° C. appears to promote the formation of fine deposits.

Example: At elevated temperatures, deposits were clearly visible after only one day at pH 8 (e.g. IS019 and IS025) and pH9 (e.g. IS033 and IS039), while at RT (IS022, IS036, IS028, IS042), virtually no deposition or no deposition was seen after one day under these conditions.

A longer embedding time should increase the amount of precipitated material, or in the case of extremely slow precipitation, a longer embedding time should be required for deposition to occur at all. Contrary to this expectation, the largest deposition amounts were observed for the samples with an embedding time of 24 h. It may be that with a longer embedding time, conversion or diffusion processes take place that decrease the visible deposits compared to samples with shorter embedding times. In-depth analyses could provide more detailed information on the distribution of elements in the mineralized substrate.

It appears at this point that a uniform coating is best achieved using the ultrasound-cleaved hyaluronic acid solution, as this solution forms a filled-in material spot on the PEEK substrate. In IS025 and IS026, preferential material deposition appears to take place on this coating. Enzymatically cleaved hyaluronic acid appears to leave only a ring of coating material on the substrate and shows no preferential material deposition areas, with the exception of a minimal area in the sample IS020.

### SUMMARY

A study was conducted on the surface functionalization of the bone implant plastic polyether ether ketone in order to allow improved incorporation into the treated bone area. Polyether ether ketone surfaces were successfully subjected to chemical modification by different methods. The surface properties were modified using small molecules, and hydroxyl, carboxyl- and imine functionalities were obtained on the surface. The modified surface was analyzed and characterized by means of ATR-IR spectroscopy (not shown). Moreover, functional polymers such as polyacrylic acid, but also polymethyl acrylate (PMA), were deposited on the polyether ether ketone surface by means of UV-induced grafting polymerization. The polyacrylic acid layer was examined by different surface analysis methods, such as ATR infrared spectroscopy, scanning electron microscopy and confocal laser scanning microscopy in order to collect spectroscopic data on the surface and obtain a precise picture of its topography (not shown). The polyacrylic acid layer was modified by coupling of diamine linkers so that amide bonds could layer be formed with organic acids. In order to allow quantitative conclusions on the degree of surface functionalization with amino groups to be drawn, the cleavable fluorescent dye C-coumarin, with which it was possible to indirectly quantitate the accessible amino groups on the surface, was synthesized. Quantitation was successfully carried out in the samples that had been directly imine-functionalized with diamines.

Hyaluronic acid with adipic acid dihydrazide, hexamethylene diamine and alginic acid was modified only with the diamine in order to couple amine linkers for subsequent anchoring to the various polyether ether ketone substrates. Hyaluronic acid was also deacetylated in order to introduce amine functionalities onto the polysaccharide in this manner. The modified polysaccharides were characterized by means of NMR and ATR infrared spectroscopic methods (not shown).

The numerous modified and unmodified polysaccharides were coupled to the complementary PEEK substrates. The coupled samples were examined by means of ATR infrared spectroscopy, scanning electron microscopy and in some cases thermogravimetry (not shown).

In the present invention, azidoaniline groups as photocoupleable or light-inducible linkers were coupled to the carboxy group of polysaccharides such as e.g. alginic or hyaluronic acid, and the latter were then bonded with light to a PEEK surface. Coating of the polyether ether ketone with azido-functionalized hyaluronic acid was successfully demonstrated. Moreover, clear indications were seen that mineralization of the PEEK surface coupled with hyaluronic acid derivatives takes place.

Outlook

As the surface-induced radical polymerization was highly successful, there are several approaches, particularly in this area, on which further studies could be based. Even though the introduction of polysaccharide structures on the polyether ether ketone surface was found not to be trivial, the polymerization with acrylic acid functioned extremely well, indicating that a highly promising approach would be to directly coat the PEEK surface with polymers of modified acrylic acid derivatives. Examples of suitable alternative acrylic-acid-based monomer units include acrylic acid derivatives modified with sugar molecules, which are known to play an important role in the cellular adhesion of osteoblasts. It would also be of interest if the monomer units carried oligosaccharides of hyaluronic acid, or also short adhesion-mediating RGD peptide sequences, etc.:

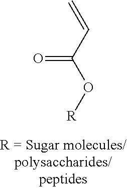

A highly sensitive surface analysis method is XPS (x-ray photoelectron spectroscopy). This method might make it possible to detect polysaccharides on the surfaces of the produced substrates.

Research on the swelling behavior of the polyacrylic acid layer on the polyether ether ketone substrate could be an approach for optimizing the coupling conditions so that it would be possible to successfully carry out polysaccharide detection even with simple analysis methods. Coupling in non-aqueous media would also be conceivable, but this approach would certainly be problematic as well due to the poor solubility of the polysaccharides.

Further studies of precipitating hydroxyapatite in the polyacrylic acid layer should also be carried out, as it has been established that the hydroxyapatite coating has positive effects on acceptance in the body.

1. A method for producing a material for a bone implant, which comprises the steps of:
providing a carrier structure having a surface formed from a biocompatible material;
coupling a covalent coupling of a matrix having at least one polysaccharide to the surface; and
mineralizing the matrix with calcium phosphate.

2. The method according to claim 1, which further comprises performing the coupling step by the following steps in any desired order:
covalent coupling of a linker molecule selected from the group consisting of a diamine linker, a diamine linker and a succinic acid linker, a UV-grafted polyacrylic acid, a photocoupleable linker, and an azidoaniline linker, to an activated surface; and
covalent coupling of the polysaccharide with carboxylic acid groups to a diamine linker molecule, or a hexamethylene-diamine-modified polysaccharide to succinic acid linkers, or an unmodified polysaccharide via ester bonds to a polyacrylic acid linker or a photocoupleable linker.

3. The method according to claim 2, which further comprises carrying out the covalent coupling of the photocoupleable linker to the activated surface at a wavelength with a range of 200 nm to 400 nm.

4. The method according to claim 2, which further comprises carrying out the covalent coupling of a carboxy-functionalized polysaccharide by means of amine and carboxyl group coupling to the photocoupleable linker.

5. The method according to claim 2, which further comprises using an azidoaniline linker as the photocoupleable linker.

6. The method according to claim 5, which further comprises carrying out the covalent coupling of the the azidoaniline linker to the activated surface at a wavelength with a range of 200 nm to 300 nm.

7. The method according to claim 3, which further comprises carrying out the covalent coupling of the photocoupleable linker to the activated surface at the wavelength with a range of 240 nm to 260 nm.

8. The method according to claim 3, which further comprises carrying out the covalent coupling of the photocoupleable linker to the activated surface at the wavelength of 254 nm.

9. The method according to claim 4, which further comprises using an azidoaniline linker as the coupleable linker.
